(19) United States
(12) Patent Application Publication
(10) Pub. No.: US 2026/0240825 A1
(43) Pub. Date: Aug. 20, 2026

(54) TOPICAL COMPOSITIONS

(71) Applicant: BAUSCH HEALTH IRELAND LIMITED, Dublin (IE)

(72) Inventors: Arturo J. ANGEL, Bridgewater, NJ (US); Radhakrishnan S. PILLAI, Bridgewater, NJ (US); Nayan DESAI, Bridgewater, NJ (US)

(21) Appl. No.: 19/472,208

(22) PCT Filed: Apr. 11, 2024
(86) PCT No.: PCT/EP2024/059822

§ 371 Date: Oct. 3, 2025

Related U.S. Application Data
(60) Provisional application No. 63/495,492, filed on Apr. 11, 2023.

(51) Int. Cl. A61K 31/439; A61K 9/00; A61K 9/107; A61K 31/01; A61K 31/05; A61K 31/07; A61K 31/125; A61K 31/138; A61K 31/167; A61K 31/17; A61K 31/19; A61K 31/192; A61K 31/23; A61K 31/245; A61K 31/327; A61K 31/4015; A61K 31/4166; A61K 31/5375; A61K 31/59; A61K 31/60; A61K 31/728; A61K 33/04; A61K 36/48; A61K 36/534; A61K 36/61; A61K 36/88; A61K 36/899; A61K 38/06; A61K 38/08; A61K 38/16; A61K 45/06; A61K 47/10; A61K 47/12; A61K 47/14; A61K 47/20; A61P 17/00

(52) U.S. Cl. CPC A61K 31/439; A61K 9/0014; A61K 9/107; A61K 31/01; A61K 31/05; A61K 31/07; A61K 31/125; A61K 31/138; A61K 31/167; A61K 31/17; A61K 31/19; A61K 31/192; A61K 31/23; A61K 31/245; A61K 31/327; A61K 31/4015; A61K 31/4166; A61K 31/5375; A61K 31/59; A61K 31/60; A61K 31/728; A61K 33/04; A61K 36/48; A61K 36/534; A61K 36/61; A61K 36/88; A61K 36/899; A61K 38/06; A61K 38/08; A61K 38/168; A61K 45/06; A61K 47/10; A61K 47/12; A61K 47/14; A61K 47/20; A61P 17/00

(57) ABSTRACT

The disclosure provides a formulation and method for treating dermatological conditions, such as atopic dermatitis, in a patient comprising administering to the affected area of a patient a topical pharmaceutical composition comprising a therapeutically active vehicle and, optionally, one or more additional active agents.

### CROSS-REFERENCE TO RELATED APPLICATIONS

This application claims the benefit of priority to U.S. Application No. 63/495,492, filed Apr. 11, 2023, the entirety of which is incorporated herein by reference.

### FIELD OF THE INVENTION

This disclosure relates to topical compositions for the treatment of dermatological conditions, such as atopic dermatitis, and more particularly, but not exclusively, to moderate to severe atopic dermatitis and methods for making and using the same.

### BACKGROUND OF THE INVENTION

Atopic dermatitis (AD) (often referred to as eczema) is a very common chronic dermatosis with a prevalence of 10% to 20% in children and 1% to 3% in adults (Leung, D Y, et al., “New Insights into Atopic Dermatitis,” 113 (5) J. Clin. Invest. 651-7 (2004)). AD poses difficult therapeutic challenges and may have a profound effect on a patient's quality of life. The disease gives rise to itching and severely dry skin, and is characterized by an allergic predisposition, pruritus, erythema, oozing, crusting, excoriations, lichenification, sensitivity to allergens/irritants, and a susceptibility to secondary infections. AD typically affects the face (cheeks), neck, arms, and legs. AD causes inflammation, redness, and irritation of the skin. It commonly presents in childhood, but can develop at any age. In most cases, there are periods of time when the disease is worse, called flares, followed by periods when the skin improves or clears up entirely, called remissions.

However, a need remains for improved treatments for moderate to severe AD, specifically for a topical treatment for moderate to severe AD.

### SUMMARY OF THE INVENTION

The disclosure provided herein describes a topical lotion comprising a therapeutically active vehicle, wherein the topical lotion is useful for treating dermatological conditions including, but not limited to, the treatment of moderate to severe AD.

In an embodiment, the disclosure provides a topical lotion comprising a therapeutically active vehicle. In some embodiments, the therapeutically active vehicle comprises a base comprising an oil phase (e.g., comprising organic solvents e.g. oleyl alcohol; medium-chain triglycerides, and combinations thereof; emulsifiers, e.g. fatty alcohols, (e.g., selected from C₁₄₋₁₈ fatty alcohols, e.g., cetyl alcohol, stearyl alcohol), mono- and di-glycerides, and combinations thereof), a water phase (e.g., comprising water and one or more anionic surfactants, e.g., selected from sodium alkyl sulfates, e.g. sodium cetostearyl sulfate), and one or more viscosity modifying agents (e.g., comprising a carbomer). In some embodiments, the therapeutically active vehicle optionally comprises a preservative (e.g., benzyl alcohol). In some embodiments, the therapeutically active vehicle comprises optionally comprises a buffer (e.g., citric acid anhydrous, sodium hydroxide, and combinations thereof).

In some embodiments, the topical lotion comprises a therapeutically active vehicle and an additional active agent. In some embodiments, the additional active agent may be selected from the group consisting of an AD agent, a natural moisturizing factor (NMF), a peptide/collagen synthesis agent, a skin rejuvenation agent, an acne agent, a skin protectant, an analgesic agent, an antipruritic agent, an antihistamine, a macrolide, and combinations thereof. In some embodiments, the additional active agent comprises a macrolide. In some embodiments, the macrolide is selected from the group consisting of rapamycin, pimecrolimus, temsirolimus, everolimus, and tacrolimus.

In some embodiments, the additional active agent is pimecrolimus. ELIDEL® (pimecrolimus) Cream, 1% was approved by the Food and Drug Administration (FDA) in 2001 as second-line therapy for the short-term and noncontinuous chronic treatment of mild to moderate AD in nonimmunocompromised adults and children 2 years of age and older who fail to respond adequately to, or are advised against using, other topical prescription treatments.

In an embodiment, the disclosure includes a method of treating a dermatological condition, such as moderate to severe AD, particularly severe AD, in a patient in need thereof, comprising administering to the affected area, e.g., once or twice daily, e.g., for a period of at least 4 weeks, e.g., at least 6 weeks, e.g., 6 weeks, a topical lotion comprising a therapeutically active vehicle and an additional active agent, for example a topical lotion comprising 0.01 to 1.5 wt. %, e.g., 1%, of the additional active agent (e.g., pimecrolimus). The therapeutically active vehicle of the topical lotion may comprise a base comprising an oil phase (e.g., comprising organic solvents e.g. oleyl alcohol; medium-chain triglycerides, and combinations thereof; emulsifiers, e.g. fatty alcohols, (e.g., selected from C₁₄₋₁₈ fatty alcohols, e.g., cetyl alcohol, stearyl alcohol), mono- and di-glycerides, and combinations thereof), a water phase (e.g., comprising water and one or more anionic surfactants, e.g., selected from sodium alkyl sulfates, e.g. sodium cetostearyl sulfate), one or more viscosity modifying agents (e.g., comprising a carbomer), optionally a preservative (e.g., benzyl alcohol), and optionally a buffer (e.g., citric acid anhydrous, sodium hydroxide, and combinations thereof).

Embodiments of the present disclosure provide a method for treating atopic dermatitis (AD) in a subject in need thereof, comprising administering to the affected area a topical pharmaceutical lotion comprising a therapeutically active vehicle, wherein the therapeutically active vehicle comprises an oil phase comprising 0 wt. % to 15 wt. % Fatty alcohols selected from Oleyl Alcohol, Cetyl Alcohol, Stearyl Alcohol, and combinations thereof; 10 wt. % to 20 wt. % Medium-chain triglycerides, and 1 wt. % to 3 wt. % Mono-glycerides and Di-glycerides; a viscosity modifying agent comprising 0.05 wt. % to 1 wt. % Carbomer Homopolymer Type A, a water phase comprising 0.5 wt. % to 2 wt. % Sodium Cetostearyl Sulfate, and 25 wt. % to 75 wt. % Water; one or more preservatives comprising 0.5 wt. % to 2 wt. % benzyl alcohol; one or more buffers comprising 0.025 wt. % to 0.2 wt. % Citric Acid, and Sodium Hydroxide, quantum satis (q.s.) to pH 5-7.5. In some embodiments, the water phase further comprises 2 wt. % to 8 wt. % propylene glycol. In some embodiments, the oil phase comprises 15 wt. % Medium-chain triglycerides. In some embodiments, the oil phase comprises 2 wt. % Mono-glycerides and Di-glycerides. In some embodiments, the viscosity modifying agent comprises 0.2 wt. % Carbomer Homopolymer Type A. In some embodiments, the water phase comprises 1 wt. % Sodium Cetostearyl Sulfate. In some embodiments, the one or more preservatives comprise 1 wt. % benzyl alcohol. In some embodiments, the one or more buffers comprise 0.05 wt. % citric acid. In some embodiments, the Sodium Hydroxide is q.s. to pH 5.5. In some embodiments, the AD is moderate to severe AD. In some embodiments, the topical pharmaceutical lotion is an emulsion. In some embodiments, the topical pharmaceutical lotion is administered to the affected area twice daily. In some embodiments, the duration of treatment is at least four weeks. In some embodiments, the duration of treatment is six weeks. In some embodiments, the atopic dermatitis is severe atopic dermatitis. In some embodiments, the subject at the start of treatment exhibits moderate to severe AD, characterized by (a) clearly perceptible erythema (dull red), clearly perceptible induration/papulation, and/or clearly perceptible lichenification, wherein oozing or crusting may be present; or (b) marked erythema (deep or bright red), marked induration/papulation, and/or marked lichenification, wherein the disease is wide-spread in extent, and wherein oozing or crusting may be present. In some embodiments, the subject at the start of treatment exhibits severe AD, characterized by marked erythema (deep or bright red), marked induration/papulation, and/or marked lichenification, wherein the disease is wide-spread in extent, and wherein oozing or crusting may be present. In some embodiments, the subject at the start of treatment has an Investigator Global Assessment (IGA) Scale score of greater than 3. In some embodiments, the subject at the start of treatment has an EASI score of greater than 21. In some embodiments, the subject exhibits at least a 2-grade reduction in the Investigator Global Assessment (IGA) Scale and exhibits an IGA that equates to “Clear” (0) or “Almost Clear” (1) after at least four weeks of treatment, e.g., after six weeks of treatment. In some embodiments, the subject exhibits 75% reduction in the Eczema Area Severity Index (EASI) score after at least four weeks of treatment, e.g., after six weeks of treatment. In some embodiments, the subject exhibits at least a 1 grade improvement from baseline in the severity of pruritus score and a pruritus score equating to “none” or “mild” after at least one week of treatment, e.g., after two weeks of treatment. In some embodiments, the topical pharmaceutical lotion comprises an additional active agent. In some embodiments, the topical pharmaceutical lotion comprises about 1-1.5 wt. % of the additional active agent. In some embodiments, the additional active agent is selected from the group consisting of an AD agent, a natural moisturizing factor (NMF), a peptide/collagen synthesis agent, a skin rejuvenation agent, an acne agent, a skin protectant, an analgesic agent, an antipruritic agent, an antihistamine, a macrolide, and combinations thereof. In some embodiments, the AD agent is Ophiopogon japonicus (Mondo grass). In some embodiments, the NMF may be selected from the group consisting of pyrrolidone carboxylic acid (PCA), hyaluronic acid, a ceramide, lactic acid, an amino acid, urea, squalene, and a combination thereof. In some embodiments, the peptide/collagen synthesis agent is selected from the group consisting of Palmitoyl Tripeptide-38, Palmitoyl Tripeptide-1, Palmitoyl Tetrapeptide-7, Acetyl hexapeptide 3, Acetyl hexapeptide 8, Acetyl hexapeptide 20, Palmitoyl oligopeptide, Lupine protein, a fractionated peptide extract (e.g., green peptides), and a combination thereof. In some embodiments, the skin rejuvenation agent is selected from the group consisting of a retinol, Retinyl Palmitate, Hydroxypinacolone Retinoate, Retinyl Lenoleate, bakuchiol extract (natural and green retinol alternative), Ceratonia siliqua (Locust bean gum), an Alpha Hydroxy Acid, a Beta Hydroxy Acid, and a combination thereof. In some embodiments, the acne agent is selected from the group consisting of Punica granatum Pericarp Extract (pomegranate peel extract), Tea tree oil, Salicylic acid, benzoyl peroxide (BPO), sulfur, and a combination thereof. In some embodiments, the skin protectant is selected from the group consisting of allantoin, calamine, and a combination thereof. In some embodiments, the analgesic agent is selected from the group consisting of lidocaine, pramoxine, benzocaine, hydrocortisone, and a combination thereof. In some embodiments, the antipruritic agent is selected from the group consisting of mint oil, menthol, camphor, Hydroxyphenyl Propamidobenzoic Acid, Avena sativa (Oat) Kernel Extract, Beta Glucan, an Avenanthramide, and combinations thereof. In some embodiments, the antihistamine is diphenylhydramine. In some embodiments, the macrolide selected from the group consisting of rapamycin, pimecrolimus, temsirolimus, everolimus, and tacrolimus. In some embodiments, the macrolide is pimecrolimus. In some embodiments, the additional active agent consists of pimecrolimus. In some embodiments, the topical pharmaceutical lotion does not include any additional active agents. In some embodiments, the topical pharmaceutical lotion consists of the therapeutically active vehicle.

Embodiments of the present disclosure provide a method for treating moderate to severe atopic dermatitis (AD) in a subject in need thereof, comprising administering to the affected area a topical pharmaceutical lotion comprising 0.5-1.5 wt. % pimecrolimus. In some embodiments, the concentration of pimecrolimus is about 1 wt. %. In some embodiments, the topical pharmaceutical lotion is in the form of an emulsion comprising an oil phase, a water phase, one or more viscosity modifying agents, optionally one or more preservatives, and optionally one or more buffers. In some embodiments, the topical pharmaceutical lotion is an emulsion lotion comprising an oil phase comprising 1.0 wt. % Pimecrolimus, 10 wt. % to 15 wt. % Fatty alcohols selected from Oleyl Alcohol, Cetyl Alcohol, Stearyl Alcohol, and combinations thereof, 10 wt. % to 20 wt. % Medium-chain triglycerides, and 1 wt. % to 3 wt. % Mono-glycerides and Di-glycerides; a viscosity modifying agent comprising 0.05 wt. % to 1 wt. % Carbomer Homopolymer Type A; a water phase comprising 0.5 wt. % to 2 wt. % Sodium Cetostearyl Sulfate, and 25 wt. % to 40 wt. % Water; one or more preservatives comprising 0.5 wt. % to 2 wt. % benzyl alcohol; one or more buffers comprising 0.025 wt. % to 0.2 wt. % Citric Acid, and Sodium Hydroxide, quantum satis (q.s.) to pH 5-7.5. In some embodiments, the water phase further comprises 2 wt. % to 8 wt. % propylene glycol. In some embodiments, the oil phase comprises 15 wt. % Medium-chain triglycerides. In some embodiments, the oil phase comprises 2 wt. % Mono-glycerides and Di-glycerides. In some embodiments, the viscosity modifying agent comprises 0.2 wt. % Carbomer Homopolymer Type A. In some embodiments, the water phase comprises 1 wt. % Sodium Cetostearyl Sulfate. In some embodiments, the one or more preservatives comprise 1 wt. % benzyl alcohol. In some embodiments, the one or more buffers comprise 0.05 wt. % citric acid. In some embodiments, the Sodium Hydroxide is q.s. to pH 5.5. In some embodiments, the topical pharmaceutical composition is administered to the affected area twice daily. In some embodiments, the duration of treatment is at least four weeks. In some embodiments, the duration of treatment is six weeks. In some embodiments, the atopic dermatitis is severe atopic dermatitis. In some embodiments, the subject at the start of treatment exhibits moderate to severe AD, characterized by (a) clearly perceptible erythema (dull red), clearly perceptible induration/papulation, and/or clearly perceptible lichenification, wherein oozing or crusting may be present; or (b) marked erythema (deep or bright red), marked induration/papulation, and/or marked lichenification, wherein the disease is wide-spread in extent, and wherein oozing or crusting may be present. In some embodiments, the subject at the start of treatment exhibits severe AD, characterized by marked erythema (deep or bright red), marked induration/papulation, and/or marked lichenification, wherein the disease is wide-spread in extent, and wherein oozing or crusting may be present. In some embodiments, the subject at the start of treatment has an Investigator Global Assessment (IGA) Scale score of greater than 3. In some embodiments, the subject at the start of treatment has an EASI score of greater than 21. In some embodiments, the subject exhibits at least a 2-grade reduction in the Investigator Global Assessment (IGA) Scale and exhibits an IGA that equates to “Clear” (0) or “Almost Clear” (1) after at least four weeks of treatment. In some embodiments, the subject exhibits at least a 2-grade reduction in the Investigator Global Assessment (IGA) Scale and exhibits an IGA that equates to “Clear” (0) or “Almost Clear” (1) after six weeks of treatment. In some embodiments, the subject exhibits 75% reduction in the Eczema Area Severity Index (EASI) score after at least four weeks of treatment. In some embodiments, the subject exhibits 75% reduction in the Eczema Area Severity Index (EASI) score after six weeks of treatment. In some embodiments, the subject exhibits at least a 1 grade improvement from baseline in the severity of pruritus score and a pruritus score equating to “none” or “mild” after at least one week of treatment. In some embodiments, the subject exhibits at least a 1 grade improvement from baseline in the severity of pruritus score and a pruritus score equating to “none” or “mild” after two weeks of treatment.

Embodiments of the present disclosure provide a topical pharmaceutical lotion comprising a therapeutically active vehicle, wherein the therapeutically active vehicle comprises an oil phase comprising 10 wt. % to 15 wt. % Fatty alcohols selected from Oleyl Alcohol, Cetyl Alcohol, Stearyl Alcohol, and combinations thereof, 10 wt. % to 20 wt. % Medium-chain triglycerides, and 1 wt. % to 3 wt. % Mono-glycerides and Di-glycerides; a viscosity modifying agent comprising 0.05 wt. % to 1 wt. % Carbomer Homopolymer Type A; a water phase comprising 0.5 wt. % to 2 wt. % Sodium Cetostearyl Sulfate, and 25 wt. % to 75 wt. % Water; one or more preservatives comprising 0.5 wt. % to 2 wt. % benzyl alcohol; one or more buffers comprising 0.025 wt. % to 0.2 wt. % Citric Acid and Sodium Hydroxide, quantum satis (q.s.) pH 5-7.5. In some embodiments, the water phase further comprises 2 wt. % to 8 wt. % propylene glycol. In some embodiments, the oil phase comprises 15 wt. % Medium-chain triglycerides. In some embodiments, the oil phase comprises 2 wt. % Mono-glycerides and Di-glycerides. In some embodiments, the viscosity modifying agent comprises 0.2 wt. % Carbomer Homopolymer Type A. In some embodiments, the water phase comprises 1 wt. % Sodium Cetostearyl Sulfate. In some embodiments, the one or more preservatives comprise 1 wt. % benzyl alcohol. In some embodiments, the one or more buffers comprise 0.05 wt. % citric acid. In some embodiments, the Sodium Hydroxide is q.s. to pH 5.5. In some embodiments, the topical pharmaceutical lotion is an emulsion. In some embodiments, the topical pharmaceutical lotion comprises an additional active agent. In some embodiments, the topical pharmaceutical lotion comprises about 1-1.5 wt. % of the additional active agent. In some embodiments, the additional active agent is selected from the group consisting of an AD agent, a natural moisturizing factor (NMF), a peptide/collagen synthesis agent, a skin rejuvenation agent, an acne agent, a skin protectant, an analgesic agent, an antipruritic agent, an antihistamine, a macrolide, and combinations thereof. In some embodiments, the AD agent is Ophiopogon japonicus (Mondo grass). In some embodiments, the NMF may be selected from the group consisting of pyrrolidone carboxylic acid (PCA), hyaluronic acid, a ceramide, lactic acid, an amino acid, urea, squalene, and a combination thereof. In some embodiments, the peptide/collagen synthesis agent is selected from the group consisting of Palmitoyl Tripeptide-38, Palmitoyl Tripeptide-1, Palmitoyl Tetrapeptide-7, Acetyl hexapeptide 3, Acetyl hexapeptide 8, Acetyl hexapeptide 20, Palmitoyl oligopeptide, Lupine protein, a fractionated peptide extract (e.g., green peptides), and a combination thereof. In some embodiments, the skin rejuvenation agent is selected from the group consisting of a retinol, Retinyl Palmitate, Hydroxypinacolone Retinoate, Retinyl Lenoleate, bakuchiol extract (natural and green retinol alternative), Ceratonia siliqua (Locust bean gum), an Alpha Hydroxy Acid, a Beta Hydroxy Acid, and a combination thereof. In some embodiments, the acne agent is selected from the group consisting of Punica granatum Pericarp Extract (pomegranate peel extract), Tea tree oil, Salicylic acid, benzoyl peroxide (BPO), sulfur, and a combination thereof. In some embodiments, the skin protectant is selected from the group consisting of allantoin, calamine, and a combination thereof. In some embodiments, the analgesic agent is selected from the group consisting of lidocaine, pramoxine, benzocaine, hydrocortisone, and a combination thereof. In some embodiments, he antipruritic agent is selected from the group consisting of mint oil, menthol, camphor, Hydroxyphenyl Propamidobenzoic Acid, Avena sativa (Oat) Kernel Extract, Beta Glucan, an Avenanthramide, and combinations thereof. In some embodiments, the antihistamine is diphenylhydramine. In some embodiments, the macrolide selected from the group consisting of rapamycin, pimecrolimus, temsirolimus, everolimus, and tacrolimus. In some embodiments, the macrolide is pimecrolimus. In some embodiments, the additional active agent consists of pimecrolimus. In some embodiments, the topical pharmaceutical lotion does not include any additional active agents. In some embodiments, the topical pharmaceutical lotion consists of the therapeutically active vehicle.

Embodiments of the present disclosure provide a method for treating atopic dermatitis (AD) in a subject in need thereof, comprising administering to the affected area a topical pharmaceutical lotion comprising a therapeutically active vehicle, wherein the therapeutically active vehicle comprises 0 wt. % to 15 wt. % Fatty alcohols selected from Oleyl Alcohol, Cetyl Alcohol, Stearyl Alcohol, and combinations thereof; 10 wt. % to 20 wt. % Medium-chain triglycerides; 1 wt. % to 3 wt. % Mono-glycerides and Di-glycerides; a viscosity modifying agent comprising 0.05 wt. % to 1 wt. % Carbomer Homopolymer Type A; 0.5 wt. % to 2 wt. % Sodium Cetostearyl Sulfate; 25 wt. % to 75 wt. % Water; one or more preservatives comprising 0.5 wt. % to 2 wt. % benzyl alcohol; one or more buffers comprising 0.025 wt. % to 0.2 wt. % Citric Acid, and Sodium Hydroxide, quantum satis (q.s.) to pH 5-7.5. In some embodiments, the topical pharmaceutical lotion further comprises 2 wt. % to 8 wt. % propylene glycol. In some embodiments, the topical pharmaceutical lotion comprises 5 wt. % Medium-chain triglycerides. In some embodiments, the topical pharmaceutical lotion comprises 2 wt. % Mono-glycerides and Di-glycerides. In some embodiments, the viscosity modifying agent comprises 0.2 wt. % Carbomer Homopolymer Type A. In some embodiments, the topical pharmaceutical lotion comprises 1 wt. % Sodium Cetostearyl Sulfate. In some embodiments, the one or more preservatives comprise 1 wt. % benzyl alcohol. In some embodiments, the one or more buffers comprise 0.05 wt. % citric acid. In some embodiments, the Sodium Hydroxide is q.s. to pH 5.5. In some embodiments, the AD is moderate to severe AD. In some embodiments, the topical pharmaceutical lotion is an emulsion. In some embodiments, the topical pharmaceutical lotion is administered to the affected area twice daily. In some embodiments, the duration of treatment is at least four weeks. In some embodiments, the duration of treatment is six weeks. In some embodiments, the atopic dermatitis is severe atopic dermatitis. In some embodiments, the subject at the start of treatment exhibits moderate to severe AD, characterized by (a) clearly perceptible erythema (dull red), clearly perceptible induration/papulation, and/or clearly perceptible lichenification, wherein oozing or crusting may be present; or (b) marked erythema (deep or bright red), marked induration/papulation, and/or marked lichenification, wherein the disease is wide-spread in extent, and wherein oozing or crusting may be present. In some embodiments, the subject at the start of treatment exhibits severe AD, characterized by marked erythema (deep or bright red), marked induration/papulation, and/or marked lichenification, wherein the disease is wide-spread in extent, and wherein oozing or crusting may be present. In some embodiments, the subject at the start of treatment has an Investigator Global Assessment (IGA) Scale score of greater than 3. In some embodiments, the subject at the start of treatment has an EASI score of greater than 21. In some embodiments, the subject exhibits at least a 2-grade reduction in the Investigator Global Assessment (IGA) Scale and exhibits an IGA that equates to “Clear” (0) or “Almost Clear” (1) after at least four weeks of treatment, e.g., after six weeks of treatment. In some embodiments, the subject exhibits 75% reduction in the Eczema Area Severity Index (EASI) score after at least four weeks of treatment, e.g., after six weeks of treatment. In some embodiments, the subject exhibits at least a 1 grade improvement from baseline in the severity of pruritus score and a pruritus score equating to “none” or “mild” after at least one week of treatment, e.g., after two weeks of treatment. In some embodiments, the topical pharmaceutical lotion comprises an additional active agent. In some embodiments, the topical pharmaceutical lotion comprises about 1-1.5 wt. % of the additional active agent. In some embodiments, the additional active agent is selected from the group consisting of an AD agent, a natural moisturizing factor (NMF), a peptide/collagen synthesis agent, a skin rejuvenation agent, an acne agent, a skin protectant, an analgesic agent, an antipruritic agent, an antihistamine, a macrolide, and combinations thereof. In some embodiments, the AD agent is Ophiopogon japonicus (Mondo grass). In some embodiments, the NMF may be selected from the group consisting of pyrrolidone carboxylic acid (PCA), hyaluronic acid, a ceramide, lactic acid, an amino acid, urea, squalene, and a combination thereof. In some embodiments, the peptide/collagen synthesis agent is selected from the group consisting of Palmitoyl Tripeptide-38, Palmitoyl Tripeptide-1, Palmitoyl Tetrapeptide-7, Acetyl hexapeptide 3, Acetyl hexapeptide 8, Acetyl hexapeptide 20, Palmitoyl oligopeptide, Lupine protein, a fractionated peptide extract (e.g., green peptides), and a combination thereof. In some embodiments, the skin rejuvenation agent is selected from the group consisting of a retinol, Retinyl Palmitate, Hydroxypinacolone Retinoate, Retinyl Lenoleate, bakuchiol extract (natural and green retinol alternative), Ceratonia siliqua (Locust bean gum), an Alpha Hydroxy Acid, a Beta Hydroxy Acid, and a combination thereof. In some embodiments, the acne agent is selected from the group consisting of Punica granatum Pericarp Extract (pomegranate peel extract), Tea tree oil, Salicylic acid, benzoyl peroxide (BPO), sulfur, and a combination thereof. In some embodiments, the skin protectant is selected from the group consisting of allantoin, calamine, and a combination thereof. In some embodiments, the analgesic agent is selected from the group consisting of lidocaine, pramoxine, benzocaine, hydrocortisone, and a combination thereof. In some embodiments, the antipruritic agent is selected from the group consisting of mint oil, menthol, camphor, Hydroxyphenyl Propamidobenzoic Acid, Avena sativa (Oat) Kernel Extract, Beta Glucan, an Avenanthramide, and combinations thereof. In some embodiments, the antihistamine is diphenylhydramine. In some embodiments, the macrolide selected from the group consisting of rapamycin, pimecrolimus, temsirolimus, everolimus, and tacrolimus. In some embodiments, the macrolide is pimecrolimus. In some embodiments, the additional active agent consists of pimecrolimus. In some embodiments, the topical pharmaceutical lotion does not include any additional active agents. In some embodiments, the topical pharmaceutical lotion is an emulsion comprising an oil phase and a water phase. In some embodiments, the topical pharmaceutical lotion consists of the therapeutically active vehicle.

Embodiments of the present disclosure provide a method for treating moderate to severe atopic dermatitis (AD) in a subject in need thereof, comprising administering to the affected area a topical pharmaceutical lotion comprising 0.5-1.5 wt. % pimecrolimus. In some embodiments, the concentration of pimecrolimus is about 1 wt. %. In some embodiments, the topical pharmaceutical lotion is in the form of an emulsion comprising an oil phase, a water phase, one or more viscosity modifying agents, optionally one or more preservatives, and optionally one or more buffers. In some embodiments, the topical pharmaceutical lotion is an emulsion lotion comprising 1.0 wt. % Pimecrolimus, 10 wt. % to 15 wt. % Fatty alcohols selected from Oleyl Alcohol, Cetyl Alcohol, Stearyl Alcohol, and combinations thereof; 10 wt. % to 20 wt. % Medium-chain triglycerides; 1 wt. % to 3 wt. % Mono-glycerides and Di-glycerides; a viscosity modifying agent comprising 0.05 wt. % to 1 wt. % Carbomer Homopolymer Type A; 0.5 wt. % to 2 wt. % Sodium Cetostearyl Sulfate; 25 wt. % to 40 wt. % Water; one or more preservatives comprising 0.5 wt. % to 2 wt. % benzyl alcohol; one or more buffers comprising 0.025 wt. % to 0.2 wt. % Citric Acid, and Sodium Hydroxide, quantum satis (q.s.) to pH 5-7.5. In some embodiments, the topical pharmaceutical lotion further comprises 2 wt. % to 8 wt. % propylene glycol. In some embodiments, the topical pharmaceutical lotion comprises 15 wt. % Medium-chain triglycerides. In some embodiments, the topical pharmaceutical lotion comprises 2 wt. % Mono-glycerides and Di-glycerides. In some embodiments, the viscosity modifying agent comprises 0.2 wt. % Carbomer Homopolymer Type A. In some embodiments, the topical pharmaceutical lotion comprises 1 wt. % Sodium Cetostearyl Sulfate. In some embodiments, the one or more preservatives comprise 1 wt. % benzyl alcohol. In some embodiments, the one or more buffers comprise 0.05 wt. % citric acid. In some embodiments, the Sodium Hydroxide is q.s. to pH 5.5. In some embodiments, the topical pharmaceutical composition is administered to the affected area twice daily. In some embodiments, the duration of treatment is at least four weeks. In some embodiments, the duration of treatment is six weeks. In some embodiments, the atopic dermatitis is severe atopic dermatitis. In some embodiments, the subject at the start of treatment exhibits moderate to severe AD, characterized by (a) clearly perceptible erythema (dull red), clearly perceptible induration/papulation, and/or clearly perceptible lichenification, wherein oozing or crusting may be present; or (b) marked erythema (deep or bright red), marked induration/papulation, and/or marked lichenification, wherein the disease is wide-spread in extent, and wherein oozing or crusting may be present. In some embodiments, the subject at the start of treatment exhibits severe AD, characterized by marked erythema (deep or bright red), marked induration/papulation, and/or marked lichenification, wherein the disease is wide-spread in extent, and wherein oozing or crusting may be present. In some embodiments, the subject at the start of treatment has an Investigator Global Assessment (IGA) Scale score of greater than 3. In some embodiments, the subject at the start of treatment has an EASI score of greater than 21. In some embodiments, the subject exhibits at least a 2-grade reduction in the Investigator Global Assessment (IGA) Scale and exhibits an IGA that equates to “Clear” (0) or “Almost Clear” (1) after at least four weeks of treatment. In some embodiments, the subject exhibits at least a 2-grade reduction in the Investigator Global Assessment (IGA) Scale and exhibits an IGA that equates to “Clear” (0) or “Almost Clear” (1) after six weeks of treatment. In some embodiments, the subject exhibits 75% reduction in the Eczema Area Severity Index (EASI) score after at least four weeks of treatment. In some embodiments, the subject exhibits 75% reduction in the Eczema Area Severity Index (EASI) score after six weeks of treatment. In some embodiments, the subject exhibits at least a 1 grade improvement from baseline in the severity of pruritus score and a pruritus score equating to “none” or “mild” after at least one week of treatment. In some embodiments, the subject exhibits at least a 1 grade improvement from baseline in the severity of pruritus score and a pruritus score equating to “none” or “mild” after two weeks of treatment.

Embodiments of the present disclosure provide a topical pharmaceutical lotion comprising a therapeutically active vehicle, wherein the therapeutically active vehicle comprises 10 wt. % to 15 wt. % Fatty alcohols selected from Oleyl Alcohol, Cetyl Alcohol, Stearyl Alcohol, and combinations thereof; 10 wt. % to 20 wt. % Medium-chain triglycerides; 1 wt. % to 3 wt. % Mono-glycerides and Di-glycerides; a viscosity modifying agent comprising 0.05 wt. % to 1 wt. % Carbomer Homopolymer Type A; 0.5 wt. % to 2 wt. % Sodium Cetostearyl Sulfate; 25 wt. % to 75 wt. % Water; one or more preservatives comprising 0.5 wt. % to 2 wt. % benzyl alcohol; one or more buffers comprising 0.025 wt. % to 0.2 wt. % Citric Acid and Sodium Hydroxide, quantum satis (q.s.) pH 5-7.5. In some embodiments, the topical pharmaceutical lotion further comprises 2 wt. % to 8 wt. % propylene glycol. In some embodiments, the topical pharmaceutical lotion comprises 15 wt. % Medium-chain triglycerides. In some embodiments, the topical pharmaceutical lotion comprises 2 wt. % Mono-glycerides and Di-glycerides. In some embodiments, the viscosity modifying agent comprises 0.2 wt. % Carbomer Homopolymer Type A. In some embodiments, the topical pharmaceutical lotion comprises 1 wt. % Sodium Cetostearyl Sulfate. In some embodiments, the one or more preservatives comprise 1 wt. % benzyl alcohol. In some embodiments, the one or more buffers comprise 0.05 wt. % citric acid. In some embodiments, the Sodium Hydroxide is q.s. to pH 5.5. In some embodiments, the topical pharmaceutical lotion is an emulsion. In some embodiments, the topical pharmaceutical lotion comprises an additional active agent. In some embodiments, the topical pharmaceutical lotion comprises about 1-1.5 wt. % of the additional active agent. In some embodiments, the additional active agent is selected from the group consisting of an AD agent, a natural moisturizing factor (NMF), a peptide/collagen synthesis agent, a skin rejuvenation agent, an acne agent, a skin protectant, an analgesic agent, an antipruritic agent, an antihistamine, a macrolide, and combinations thereof. In some embodiments, the AD agent is Ophiopogon japonicus (Mondo grass). In some embodiments, the NMF may be selected from the group consisting of pyrrolidone carboxylic acid (PCA), hyaluronic acid, a ceramide, lactic acid, an amino acid, urea, squalene, and a combination thereof. In some embodiments, the peptide/collagen synthesis agent is selected from the group consisting of Palmitoyl Tripeptide-38, Palmitoyl Tripeptide-1, Palmitoyl Tetrapeptide-7, Acetyl hexapeptide 3, Acetyl hexapeptide 8, Acetyl hexapeptide 20, Palmitoyl oligopeptide, Lupine protein, a fractionated peptide extract (e.g., green peptides), and a combination thereof. In some embodiments, the skin rejuvenation agent is selected from the group consisting of a retinol, Retinyl Palmitate, Hydroxypinacolone Retinoate, Retinyl Lenoleate, bakuchiol extract (natural and green retinol alternative), Ceratonia siliqua (Locust bean gum), an Alpha Hydroxy Acid, a Beta Hydroxy Acid, and a combination thereof. In some embodiments, the acne agent is selected from the group consisting of Punica granatum Pericarp Extract (pomegranate peel extract), Tea tree oil, Salicylic acid, benzoyl peroxide (BPO), sulfur, and a combination thereof. In some embodiments, the skin protectant is selected from the group consisting of allantoin, calamine, and a combination thereof. In some embodiments, the analgesic agent is selected from the group consisting of lidocaine, pramoxine, benzocaine, hydrocortisone, and a combination thereof. In some embodiments, he antipruritic agent is selected from the group consisting of mint oil, menthol, camphor, Hydroxyphenyl Propamidobenzoic Acid, Avena sativa (Oat) Kernel Extract, Beta Glucan, an Avenanthramide, and combinations thereof. In some embodiments, the antihistamine is diphenylhydramine. In some embodiments, the macrolide selected from the group consisting of rapamycin, pimecrolimus, temsirolimus, everolimus, and tacrolimus. In some embodiments, the macrolide is pimecrolimus. In some embodiments, the additional active agent consists of pimecrolimus. In some embodiments, the topical pharmaceutical lotion does not include any additional active agents. In some embodiments, the topical pharmaceutical lotion consists of the therapeutically active vehicle. In some embodiments, the topical pharmaceutical lotion is an emulsion. In some embodiments, the topical pharmaceutical lotion comprises an oil phase and a water phase.

Further areas of applicability of the disclosure will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION OF THE INVENTION

Unless defined otherwise, all technical and scientific terms used herein have the same meaning as is commonly understood by a person having ordinary skill in the art to which this invention pertains. All patents and publications referred to herein are incorporated by reference in their entireties.

Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition, method or process that “consist of” or “consist essentially of” the described features.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments described herein that encompass the invention can, in alternate embodiments, be more specifically defined by any of the transitional terms “comprising,” “consisting essentially of,” and “consisting of.”

The topical lotions described herein may be used for the treatment of dermatological disease including, but not limited to, atopic dermatitis. In some embodiments, a topical lotion described herein can be used for the treatment of atopic dermatitis (AD). In some embodiments, a topical lotion described herein can be used for the treatment of moderate to severe AD.

In an embodiment, the topical lotion described herein comprising a therapeutically active vehicle, and optionally an additional active agent, can be administered to patients suffering from a dermatological disease. As used herein a “therapeutically active vehicle” refers to a combination of two or more pharmaceutical excipients that do not independently, but rather collectively, elicit a meaningful therapeutic response in a patient suffering from a dermatological condition, such as AD. A “therapeutically active vehicle” may therefore be distinguished from an “active agent,” as used herein, since an active agent is understood to be a compound that is known to independently elicit a therapeutic response in a patient.

In some embodiments, the topical lotion described herein comprising a therapeutically active vehicle, and optionally an additional active agent, can be administered to patients suffering from AD. In some embodiments, the topical lotion described herein comprising a therapeutically active vehicle, and optionally an additional active agent, can be administered to patients suffering from moderate to severe AD.

In some embodiments, the topical lotion described herein comprising a therapeutically active vehicle, and optionally an additional active agent, can be administered to patients suffering from a dermatological condition by application of a thin layer of the topical lotion twice daily at approximately the same time each day and approximately 12 hours apart to affected areas of the skin. The duration of treatment may be at least 4 weeks. In some embodiments, the duration of treatment is 4-10 weeks. In some embodiments, the duration of treatment is about 6 weeks. In some embodiments, the duration of treatment is about 1 week. In some embodiments, the duration of treatment is about 2 weeks. In some embodiments, the duration of treatment is about 3 weeks. In some embodiments, the duration of treatment is about 4 weeks. In some embodiments, the duration of treatment is about 5 weeks. In some embodiments, the duration of treatment is about 7 weeks. In some embodiments, the duration of treatment is about 8 weeks. In some embodiments, the duration of treatment is about 9 weeks. In some embodiments, the duration of treatment is about 10 weeks. In some embodiments, the duration of treatment is about 11 weeks. In some embodiments, the duration of treatment is about 12 weeks.

In some embodiments, the topical lotion described herein comprising a therapeutically active vehicle, and optionally an additional active agent, can be administered to patients suffering from AD by application of a thin layer of the topical lotion twice daily at approximately the same time each day and approximately 12 hours apart to affected areas of the skin. The duration of treatment may be at least 4 weeks. In some embodiments, the duration of treatment is 4-10 weeks. In some embodiments, the duration of treatment is about 6 weeks. In some embodiments, the duration of treatment is about 6 weeks. In some embodiments, the duration of treatment is about 1 week. In some embodiments, the duration of treatment is about 2 weeks. In some embodiments, the duration of treatment is about 3 weeks. In some embodiments, the duration of treatment is about 4 weeks. In some embodiments, the duration of treatment is about 5 weeks. In some embodiments, the duration of treatment is about 7 weeks. In some embodiments, the duration of treatment is about 8 weeks. In some embodiments, the duration of treatment is about 9 weeks. In some embodiments, the duration of treatment is about 10 weeks. In some embodiments, the duration of treatment is about 11 weeks. In some embodiments, the duration of treatment is about 12 weeks.

In some embodiments, the topical lotion described herein comprising a therapeutically active vehicle, and optionally an additional active agent, can be administered to patients suffering from moderate to severe AD by application of a thin layer of the topical lotion twice daily at approximately the same time each day and approximately 12 hours apart to affected areas of the skin. The duration of treatment may be at least 4 weeks. In some embodiments, the duration of treatment is 4-10 weeks. In some embodiments, the duration of treatment is about 6 weeks. In some embodiments, the duration of treatment is about 6 weeks. In some embodiments, the duration of treatment is about 1 week. In some embodiments, the duration of treatment is about 2 weeks. In some embodiments, the duration of treatment is about 3 weeks. In some embodiments, the duration of treatment is about 4 weeks. In some embodiments, the duration of treatment is about 5 weeks. In some embodiments, the duration of treatment is about 7 weeks. In some embodiments, the duration of treatment is about 8 weeks. In some embodiments, the duration of treatment is about 9 weeks. In some embodiments, the duration of treatment is about 10 weeks. In some embodiments, the duration of treatment is about 11 weeks. In some embodiments, the duration of treatment is about 12 weeks.

In an embodiment, the topical lotion described herein comprising a therapeutically active vehicle and an additional active agent, can be administered to patients suffering from a dermatological condition. In some embodiments, the topical lotion described herein comprising a therapeutically active vehicle and an additional active agent, can be administered to patients suffering from AD. In some embodiments, the topical lotion described herein comprising a therapeutically active vehicle and an additional active agent, can be administered to patients suffering from moderate to severe AD.

In some embodiments, the topical lotion described herein comprising a therapeutically active vehicle and an additional active agent, can be administered to patients suffering from a dermatological condition by application of a thin layer of the topical lotion twice daily at approximately the same time each day and approximately 12 hours apart to affected areas of the skin. The duration of treatment may be at least 4 weeks. In some embodiments, the duration of treatment is 4-10 weeks. In some embodiments, the duration of treatment is about 6 weeks. In some embodiments, the duration of treatment is about 6 weeks. In some embodiments, the duration of treatment is about 1 week. In some embodiments, the duration of treatment is about 2 weeks. In some embodiments, the duration of treatment is about 3 weeks. In some embodiments, the duration of treatment is about 4 weeks. In some embodiments, the duration of treatment is about 5 weeks. In some embodiments, the duration of treatment is about 7 weeks. In some embodiments, the duration of treatment is about 8 weeks. In some embodiments, the duration of treatment is about 9 weeks. In some embodiments, the duration of treatment is about 10 weeks. In some embodiments, the duration of treatment is about 11 weeks. In some embodiments, the duration of treatment is about 12 weeks.

In some embodiments, the topical lotion described herein comprising a therapeutically active vehicle and an additional active agent, can be administered to patients suffering from AD by application of a thin layer of the topical lotion twice daily at approximately the same time each day and approximately 12 hours apart to affected areas of the skin. The duration of treatment may be at least 4 weeks. In some embodiments, the duration of treatment is 4-10 weeks. In some embodiments, the duration of treatment is about 6 weeks. In some embodiments, the duration of treatment is about 6 weeks. In some embodiments, the duration of treatment is about 1 week. In some embodiments, the duration of treatment is about 2 weeks. In some embodiments, the duration of treatment is about 3 weeks. In some embodiments, the duration of treatment is about 4 weeks. In some embodiments, the duration of treatment is about 5 weeks. In some embodiments, the duration of treatment is about 7 weeks. In some embodiments, the duration of treatment is about 8 weeks. In some embodiments, the duration of treatment is about 9 weeks. In some embodiments, the duration of treatment is about 10 weeks. In some embodiments, the duration of treatment is about 11 weeks. In some embodiments, the duration of treatment is about 12 weeks.

In some embodiments, the topical lotion described herein comprising a therapeutically active vehicle and an additional active agent, can be administered to patients suffering from moderate to severe AD by application of a thin layer of the topical lotion twice daily at approximately the same time each day and approximately 12 hours apart to affected areas of the skin. The duration of treatment may be at least 4 weeks. In some embodiments, the duration of treatment is 4-10 weeks. In some embodiments, the duration of treatment is about 6 weeks. In some embodiments, the duration of treatment is about 6 weeks. In some embodiments, the duration of treatment is about 1 week. In some embodiments, the duration of treatment is about 2 weeks. In some embodiments, the duration of treatment is about 3 weeks. In some embodiments, the duration of treatment is about 4 weeks. In some embodiments, the duration of treatment is about 5 weeks. In some embodiments, the duration of treatment is about 7 weeks. In some embodiments, the duration of treatment is about 8 weeks. In some embodiments, the duration of treatment is about 9 weeks. In some embodiments, the duration of treatment is about 10 weeks. In some embodiments, the duration of treatment is about 11 weeks. In some embodiments, the duration of treatment is about 12 weeks.

In some embodiments of the foregoing topical lotion, the therapeutically active vehicle comprises an oil phase, a water phase, and one more viscosity modifying agents. In some embodiments of the foregoing topical lotion, the topical lotion is an emulsion.

In some embodiments of the foregoing topical lotion, the topical lotion is an emulsion and comprises an oil phase, a water phase, and one more viscosity modifying agents.

In some embodiments of the foregoing topical lotion, the topical lotion is an emulsion and comprising an oil phase, a water phase, and one or more viscosity modifying agents, wherein the oil phase comprises medium-chain triglycerides, one or more emulsifiers, wherein the emulsifiers are selected from mono-glycerides and di-glycerides, fatty alcohols and combinations thereof.

In some embodiments of the foregoing topical lotion, the topical lotion is an emulsion comprising an oil phase, a water phase, and one or more viscosity modifying agents, wherein the oil phase comprises medium-chain triglycerides, one or more emulsifiers, wherein the emulsifiers are selected from mono- and di-glycerides, fatty alcohols and combinations thereof, wherein the emulsifiers comprise fatty alcohols selected from C₁₄₋₁₈ fatty alcohols, e.g, cetyl alcohol, stearyl alcohol, and combinations thereof.

In some embodiments of the foregoing topical lotion, the topical lotion is an emulsion comprising an oil phase, a water phase, and one or more viscosity modifying agents, wherein the oil phase comprises medium-chain triglycerides, one or more emulsifiers, wherein the emulsifiers are selected from mono- and di-glycerides, fatty alcohols and combinations thereof, wherein the emulsifiers comprise mono- and di-glycerides, cetyl alcohol, and stearyl alcohol.

In some embodiments of the foregoing topical lotion, the oil phase comprises 10 wt. % to 60 wt. % of the formulation by weight. In some embodiments of the foregoing topical lotion, the oil phase comprises 25 wt. % to 45 wt. % of the formulation by weight. In some embodiments of the foregoing topical lotion, the oil phase comprises 10 wt. % to 20 wt. %, 20 wt. % to 30 wt. %, 30 wt. % to 40 wt. %, 40 wt. % to 50 wt. %, or 50 wt. % to 60 wt. %, of the formulation by weight. In some embodiments of the foregoing topical lotion, the oil phase comprises about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, about 50 wt. %, about 51 wt. %, about 52 wt. %, about 53 wt. %, about 54 wt. %, about 55 wt. %, about 56 wt. %, about 57 wt. %, about 58 wt. %, about 59 wt. %, or about 60 wt. % of the formulation by weight.

In some embodiments of the foregoing topical lotion, the topical lotion is an emulsion comprising an oil phase, a water phase, and one or more viscosity modifying agents. In some embodiments, the water phase comprises water and one or more anionic surfactants. In some embodiments, the one or more anionic surfactants are selected from sodium alkyl sulfates and combinations thereof. In some embodiments, the sodium alkyl sulfates is sodium cetostearyl sulfate.

In some embodiments of the foregoing topical lotion, the topical lotion is an emulsion comprising an oil phase, a water phase, and one or more viscosity modifying agents, wherein the water phase comprises water and one or more anionic surfactants, wherein the one or more anionic surfactants comprise sodium cetostearyl sulfate.

In some embodiments of the foregoing topical lotion, the topical lotion is an emulsion comprising an oil phase, a water phase, and one or more viscosity modifying agents, wherein the one or more viscosity modifying agents comprise a carbomer. The water phase includes an amount of water that varies depending upon the concentration of other elements in the topical lotion. In some embodiments, the water phase of the topical lotion contains 25 wt. % to 75 wt. % water. In some embodiments where the topical lotion comprises both a therapeutically active vehicle and an additional active agent, the water phase of the topical lotion may contain 25 wt. % to 40 wt. % water. In some embodiments where the topical lotion comprises a therapeutically active vehicle and does not include an additional active agent, the water phase of the topical lotion may contain 55 wt. % to 75 wt. % water.

In some embodiments of the foregoing topical lotion, the topical lotion is an emulsion comprising an oil phase, a water phase, and one or more viscosity modifying agents, wherein the one or more viscosity modifying agents comprise a carbomer, wherein the carbomer is a carbomer homopolymer Type A (polyacrylate cross-linked with allyl pentaerythritol), e.g., as defined by the United States Pharmacopeia/National Formulary (USP/NF) monograph. In some embodiments, the carbomer is a benzene-free carbomer homopolymer Type A, e.g. Carbopol 981.

In some embodiments of the foregoing topical lotion, the topical lotion is an emulsion further comprising one or more preservatives. In some embodiments, the one or more preservatives comprise benzyl alcohol.

In some embodiments of the foregoing topical lotion, the topical lotion is an emulsion further comprising one or more buffers. In some embodiments, the one or more buffers comprise citric acid anhydrous, sodium hydroxide, or combinations thereof.

In some embodiments of the foregoing topical lotion, the pH of the topical lotion is 5-7.5. In some embodiments of the foregoing topical lotion, the pH of the topical lotion is 5-6. In some embodiments of the foregoing topical lotion, the pH of the topical lotion is pH 5.1. In some embodiments of the foregoing topical lotion, the pH of the topical lotion is pH 5.2. In some embodiments of the foregoing topical lotion, the pH of the topical lotion is pH 5.3. In some embodiments of the foregoing topical lotion, the pH of the topical lotion is pH 5.4. In some embodiments of the foregoing topical lotion, the pH of the topical lotion is pH 5.5. In some embodiments of the foregoing topical lotion, the pH of the topical lotion is pH 5.6. In some embodiments of the foregoing topical lotion, the pH of the topical lotion is pH 5.7. In some embodiments of the foregoing topical lotion, the pH of the topical lotion is pH 5.8. In some embodiments of the foregoing topical lotion, the pH of the topical lotion is pH 5.9. In some embodiments of the foregoing topical lotion, the pH of the topical lotion is pH 6.0. In some embodiments of the foregoing topical lotion, the pH of the topical lotion is pH 6.1. In some embodiments of the foregoing topical lotion, the pH of the topical lotion is pH 6.2. In some embodiments of the foregoing topical lotion, the pH of the topical lotion is pH 6.3. In some embodiments of the foregoing topical lotion, the pH of the topical lotion is pH 6.4. In some embodiments of the foregoing topical lotion, the pH of the topical lotion is pH 6.5. In some embodiments of the foregoing topical lotion, the pH of the topical lotion is pH 6.6. In some embodiments of the foregoing topical lotion, the pH of the topical lotion is pH 6.7. In some embodiments of the foregoing topical lotion, the pH of the topical lotion is pH 6.8. In some embodiments of the foregoing topical lotion, the pH of the topical lotion is pH 6.9. In some embodiments of the foregoing topical lotion, the pH of the topical lotion is pH 7.0. In some embodiments of the foregoing topical lotion, the pH of the topical lotion is pH 7.1. In some embodiments of the foregoing topical lotion, the pH of the topical lotion is pH 7.2. In some embodiments of the foregoing topical lotion, the pH of the topical lotion is pH 7.3. In some embodiments of the foregoing topical lotion, the pH of the topical lotion is pH 7.4. In some embodiments of the foregoing topical lotion, the pH of the topical lotion is pH 7.5.

In an embodiment, the topical lotion described herein is an emulsion comprising a therapeutically active vehicle, wherein the therapeutically active vehicle comprises:
an oil phase comprising:
   10 wt. % to 15 wt. % Fatty alcohols selected from Oleyl Alcohol, Cetyl Alcohol, Stearyl Alcohol, and combinations thereof;
   10 wt. % to 20 wt. % Medium-chain triglycerides; and
   1 wt. % to 3 wt. % Mono-glycerides and Di-glycerides;
a viscosity modifying agent comprising 0.05 wt. % to 1 wt. % Carbomer Homopolymer Type A;
a water phase comprising:
   0.5 wt. % to 2 wt. % Sodium Cetostearyl Sulfate; and
   2 wt. % to 8 wt. % propylene glycol; and
   25 wt. % to 75 wt. % Water;
one or more preservatives comprising 0.5 wt. % to 2 wt. % benzyl alcohol;
one or more buffers comprising:
   0.025 wt. % to 0.2 wt. % Citric Acid; and
Sodium Hydroxide, quantum satis (q.s.) pH 5-7.5.

In an embodiment, the topical lotion described herein is an emulsion comprising a therapeutically active vehicle, wherein the therapeutically active vehicle comprises:
an oil phase comprising:
   10 wt. % to 15 wt. % Fatty alcohols selected from Oleyl Alcohol, Cetyl Alcohol, Stearyl Alcohol, and combinations thereof;
   10 wt. % to 20 wt. % Medium-chain triglycerides; and
   1 wt. % to 3 wt. % Mono-glycerides and Di-glycerides;
a viscosity modifying agent comprising 0.05 wt. % to 1 wt. % Carbomer Homopolymer Type A;
a water phase comprising:
   0.5 wt. % to 2 wt. % Sodium Cetostearyl Sulfate;
   2 wt. % to 8 wt. % propylene glycol; and
   25 wt. % to 75 wt. % Water;
one or more preservatives comprising 0.5 wt. % to 2 wt. % benzyl alcohol;
one or more buffers comprising:
   0.025 wt. % to 0.2 wt. % Citric Acid; and
Sodium Hydroxide, quantum satis (q.s.) pH 5-6.

In an embodiment, the topical lotion described herein is an emulsion comprising a therapeutically active vehicle, wherein the topical lotion does not contain an additional active agent, the therapeutically active vehicle comprises:
an oil phase comprising:
   10 wt. % to 15 wt. % Fatty alcohols selected from Oleyl Alcohol, Cetyl Alcohol, Stearyl Alcohol, and combinations thereof;
   10 wt. % to 20 wt. % Medium-chain triglycerides; and
   1 wt. % to 3 wt. % Mono-glycerides and Di-glycerides;
a viscosity modifying agent comprising 0.05 wt. % to 1 wt. % Carbomer Homopolymer Type A;
a water phase comprising:
   0.5 wt. % to 2 wt. % Sodium Cetostearyl Sulfate; and
   2 wt. % to 8 wt. % propylene glycol; and
   55 wt. % to 75 wt. % Water;
one or more preservatives comprising 0.5 wt. % to 2 wt. % benzyl alcohol;
one or more buffers comprising:
   0.025 wt. % to 0.2 wt. % Citric Acid; and
Sodium Hydroxide, quantum satis (q.s.) pH 5-7.5.

In an embodiment, the topical lotion described herein is an emulsion comprising a therapeutically active vehicle, wherein the topical lotion does not contain an additional active agent, the therapeutically active vehicle comprises:
an oil phase comprising:
   10 wt. % to 15 wt. % Fatty alcohols selected from Oleyl Alcohol, Cetyl Alcohol, Stearyl Alcohol, and combinations thereof;
   10 wt. % to 20 wt. % Medium-chain triglycerides; and
   1 wt. % to 3 wt. % Mono-glycerides and Di-glycerides;
a viscosity modifying agent comprising 0.05 wt. % to 1 wt. % Carbomer Homopolymer Type A;
a water phase comprising:
   0.5 wt. % to 2 wt. % Sodium Cetostearyl Sulfate;
   2 wt. % to 8 wt. % propylene glycol; and
   55 wt. % to 75 wt. % Water;
one or more preservatives comprising 0.5 wt. % to 2 wt. % benzyl alcohol;
one or more buffers comprising:
   0.025 wt. % to 0.2 wt. % Citric Acid; and
Sodium Hydroxide, quantum satis (q.s.) pH 5-6.

In an embodiment, the topical lotion described herein is an emulsion comprising a therapeutically active vehicle, wherein the therapeutically active vehicle comprises: an oil phase comprising: 10 wt. % to 15 wt. % Fatty alcohols selected from Oleyl Alcohol, Cetyl Alcohol, Stearyl Alcohol, and combinations thereof; 10 wt. % to 20 wt. % Medium-chain triglycerides: 10-20%, e.g., ca. 15%; and 1 wt. % to 3 wt. % Mono-glycerides and Di-glycerides; a viscosity modifying agent comprising 0.05 wt. % to 1 wt. % Carbomer Homopolymer Type A; a water phase comprising: 0.5 wt. % to 2 wt. % Sodium Cetostearyl Sulfate; 2 wt. % to 8 wt. % propylene glycol; and 25 wt. % to 75 wt. % Water; one or more preservatives comprising 0.5 wt. % to 2 wt. % benzyl alcohol; one or more buffers comprising: 0.025 wt. % to 0.2 wt. % Citric Acid; and Sodium Hydroxide, quantum satis (q.s.) pH 5-7.5.

In an embodiment, the topical lotion described herein is an emulsion comprising a therapeutically active vehicle, wherein the therapeutically active vehicle comprises: an oil phase comprising: 10 wt. % to 15 wt. % Fatty alcohols selected from Oleyl Alcohol, Cetyl Alcohol, Stearyl Alcohol, and combinations thereof; 10 wt. % to 20 wt. % Medium-chain triglycerides: 10-20%, e.g., ca. 15%; and 1 wt. % to 3 wt. % Mono-glycerides and Di-glycerides; a viscosity modifying agent comprising 0.05 wt. % to 1 wt. % Carbomer Homopolymer Type A; a water phase comprising: 0.5 wt. % to 2 wt. % Sodium Cetostearyl Sulfate; 2 wt. % to 8 wt. % propylene glycol; and 55 wt. % to 75 wt. % Water; one or more preservatives comprising 0.5 wt. % to 2 wt. % benzyl alcohol; one or more buffers comprising: 0.025 wt. % to 0.2 wt. % Citric Acid; and Sodium Hydroxide, quantum satis (q.s.) pH 5-7.5.

In an embodiment, the topical lotion described herein is an emulsion comprising a therapeutically active vehicle, wherein the therapeutically active vehicle comprises: an oil phase comprising: 10 wt. % to 15 wt. % Fatty alcohols selected from Oleyl Alcohol, Cetyl Alcohol, Stearyl Alcohol, and combinations thereof; 10 wt. % to 20 wt. % Medium-chain triglycerides: 10-20%, e.g., ca. 15%; and 1 wt. % to 3 wt. % Mono-glycerides and Di-glycerides; a viscosity modifying agent comprising 0.05 wt. % to 1 wt. % Carbomer Homopolymer Type A; a water phase comprising: 0.5 wt. % to 2 wt. % Sodium Cetostearyl Sulfate; 2 wt. % to 8 wt. % propylene glycol; and 25 wt. % to 40 wt. % Water; one or more preservatives comprising 0.5 wt. % to 2 wt. % benzyl alcohol; one or more buffers comprising: 0.025 wt. % to 0.2 wt. % Citric Acid; and Sodium Hydroxide, quantum satis (q.s.) pH 5-7.5.

In an embodiment, the topical lotion described herein is an emulsion comprising:

| Component | Concentration (wt. %) w/w) |
|---|---|
| Caprylic/Capric Triglycerides | 13-17 |
| Oleyl Alcohol | 8-12 |
| Sodium Cetostearyl Sulfate | 0.5-1.5 |
| Mono- and Di-glyceride | 1-3 |
| Carbopol 981 | 0.1-0.5 |
| (Carbomer Homopolymer Type A) | |
| Cetyl Alcohol | 0.5-1.5 |
| Stearyl Alcohol | 0.5-1.5 |
| Benzyl Alcohol | 0.5-1.5 |
| Propylene Glycol | 2-8 |
| Citric Acid, Anhydrous | 0.03-0.1 |
| Sodium Hydroxide | q.s. pH 5-7.5 |
| Purified water | Balance |

In some embodiments, a topical lotion described herein is an emulsion comprising:

| Component | Concentration (wt. %) w/w) |
|---|---|
| Caprylic/Capric Triglycerides | 13-14, 14-15, 15-16, or 16-17 |
| Oleyl Alcohol | 8-9, 9-10, 10-11, or 11-12 |
| Sodium Cetostearyl Sulfate | 0.5-1.0, or 1.0-1.5 |
| Mono- and Di-glyceride | 12, or 2-3 |
| Carbopol 981 | 0.1-0.2, 0.2-0.3, 0.3-0.4, or 0.4-0.5 |
| (Carbomer Homopolymer Type A) | |
| Cetyl Alcohol | 0.5-0.8, 0.8-1.0, 1.0-1.2, or 1.2-1.5 |
| Stearyl Alcohol | 0.5-0.8, 0.8-1.0, 1.0-1.2, or 1.2-1.5 |
| Benzyl Alcohol | 0.5-0.8, 0.8-1.0, 1.0-1.2, or 1.2-1.5 |
| Propylene Glycol | 2-3, 3-4, 4-5, 5-6, 6-7, or 7-8 |
| Citric Acid, Anhydrous | 0.03-0.05, 0.05-0.07, or 0.07-0.1 |
| Sodium Hydroxide | q.s. pH 5-5.5, or 5.5-6 |
| Purified water | Balance |

In an embodiment, the topical lotion described herein is an emulsion comprising:

| Component | Concentration (wt. %) w/w) |
|---|---|
| Caprylic/Capric Triglycerides | 15 |
| Oleyl Alcohol | 10 |
| Sodium Cetostearyl Sulfate | 1.0 |
| Mono- and Di-glyceride | 2.0 |
| Carbopol 981 | 0.2 |
| (Carbomer Homopolymer Type A) | |
| Cetyl Alcohol | 1.0 |
| Stearyl Alcohol | 1.0 |
| Benzyl Alcohol | 1.0 |
| Propylene Glycol | 5.0 |
| Citric Acid, Anhydrous | 0.05 |
| Sodium Hydroxide | q.s. pH 5.5 |
| Purified water | Balance |

In some embodiments, the topical lotion comprises a therapeutically active vehicle and an additional active agent, wherein the therapeutically active vehicle comprises an oil phase, a water phase, and one more viscosity modifying agents. In some embodiments, the topical lotion is an emulsion.

In some embodiments, the topical lotion comprises a therapeutically active vehicle and an additional active agent, wherein the therapeutically active vehicle provides an emulsion and comprises an oil phase, a water phase, and one more viscosity modifying agents.

In some embodiments, the topical lotion comprises a therapeutically active vehicle and an additional active agent, wherein the therapeutically active vehicle provides an emulsion and comprising an oil phase, a water phase, and one or more viscosity modifying agents, wherein the oil phase comprises medium-chain triglycerides, one or more emulsifiers, wherein the emulsifiers are selected from mono- and di-glycerides, fatty alcohols and combinations thereof.

In some embodiments, the topical lotion comprises a therapeutically active vehicle and an additional active agent, wherein the therapeutically active vehicle provides an emulsion comprising an oil phase, a water phase, and one or more viscosity modifying agents. In some embodiments, the oil phase comprises medium-chain triglycerides. In some embodiments, the oil phase comprises one or more emulsifiers. In some embodiments, the emulsifiers are selected from mono-glycerides and di-glycerides, fatty alcohols and combinations thereof. In some embodiments, the emulsifiers comprise fatty alcohols selected from C₁₄₋₁₈ fatty alcohols. In some embodiments, the fatty alcohols comprise cetyl alcohol, stearyl alcohol, and combinations thereof. In some embodiments, the topical lotion comprises 10 wt. %, 11 wt. %, 12 wt. %, 13 wt. %, 14 wt. %, or 15 wt. % fatty alcohols. In some embodiments, the topical lotion comprises 10 wt. %, 11 wt. %, 12 wt. %, 13 wt. %, 14 wt. %, 15 wt. %, 16 wt. %, 17 wt. %, 18 wt. %, 19 wt. %, or 20 wt. % Medium-chain triglycerides. In some embodiments, the topical lotion comprises 1 wt. %, 2 wt. %, 3 wt. %, 4 wt. %, or 5 wt. % Mono-glycerides and/or Di-glycerides.

In some embodiments, the topical lotion comprises a therapeutically active vehicle and an additional active agent, wherein the therapeutically active vehicle provides an emulsion comprising an oil phase, a water phase, and one or more viscosity modifying agents, wherein the oil phase comprises medium-chain triglycerides, one or more emulsifiers, wherein the emulsifiers are selected from mono- and di-glycerides, fatty alcohols and combinations thereof, wherein the emulsifiers comprise mono-glycerides and di-glycerides, cetyl alcohol, and stearyl alcohol.

In some embodiments, the topical lotion comprises a therapeutically active vehicle and an additional active agent, wherein the therapeutically active vehicle provides an emulsion comprising an oil phase, a water phase, and one or more viscosity modifying agents. In some embodiments, the water phase comprises water and one or more anionic surfactants. In some embodiments, the anionic surfactant is a sodium alkyl sulfate. In some embodiments, the anionic surfactant comprises sodium cetostearyl sulfate. In some embodiments, the topical lotion comprises 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, or 2 wt. % sodium cetostearyl sulfate.

In some embodiments, the topical lotion comprises a therapeutically active vehicle and an additional active agent, wherein the therapeutically active vehicle provides an emulsion comprising an oil phase, a water phase, and one or more viscosity modifying agents, wherein the one or more viscosity modifying agents comprise a carbomer. In some embodiments, the viscosity modifying agent is Carbomer Homopolymer Type A.

In some embodiments, the topical lotion comprises a therapeutically active vehicle and an additional active agent, wherein the therapeutically active vehicle provides an emulsion comprising an oil phase, a water phase, and one or more viscosity modifying agents, wherein the one or more viscosity modifying agents comprise a carbomer. In some embodiments, the carbomer is a carbomer homopolymer Type A (polyacrylate cross-linked with allyl pentaerythritol), e.g., as defined by the United States Pharmacopeia/National Formulary (USP/NF) monograph. In some embodiments, the carbomer is a benzene-free carbomer homopolymer Type A. In some embodiments, the carbomer is Carbopol 981. In some embodiments, the topical lotion comprises 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, or 1.5 wt. % Carbomer Homopolymer Type A.

In some embodiments, the topical lotion comprises a therapeutically active vehicle and an additional active agent, wherein the therapeutically active vehicle provides an emulsion further comprising one or more preservatives. In some embodiments, the one or more preservatives comprise benzyl alcohol. In some embodiments, the topical lotion comprises 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, or 2 wt. % benzyl alcohol.

In some embodiments, the topical lotion comprises a therapeutically active vehicle and an additional active agent, wherein the therapeutically active vehicle provides an emulsion further comprising one or more buffers. In some embodiments, the one or more buffers comprise citric acid anhydrous, sodium hydroxide, or combinations thereof. In some embodiments, the topical lotion comprises 0.02 wt. %, 0.025 wt. %, 0.03 wt. %, 0.035 wt. %, 0.04 wt. %, 0.045 wt. %, 0.05 wt. %, 0.055 wt. %, 0.06 wt. %, 0.065 wt. %, 0.07 wt. %, 0.075 wt. %, 0.08 wt. %, 0.085 wt. %, 0.09 wt. %, 0.095 wt. %, 0.1 wt. %, 0.15 wt. %, 0.2 wt. %, 0.25 wt. %, 0.3 wt. %, 0.35 wt. %, 0.4 wt. %, 0.45 wt. %, 0.5 wt. %, 0.55 wt. %, 0.6 wt. %, 0.65 wt. %, 0.7 wt. %, 0.8 wt. %, 0.85 wt. %, 0.9 wt. %, 0.95 wt. %, or 0.2 wt. % buffers.

In some embodiments of the foregoing topical lotion, the pH of the topical lotion is 5-7.5. In some embodiments, the pH of the topical lotion is 4, 4.5, 5, 5.5, 6, or 6.5.

In some embodiments of the foregoing topical lotion, the oil phase comprises 10-60% of the formulation by weight. In some embodiments, the oil phase comprises 25-45% of the formulation by weight.

In some embodiments, the therapeutically active vehicle comprises an oil phase comprising 10 wt. %-15 wt. % fatty alcohols selected from Oleyl Alcohol, Cetyl Alcohol, Stearyl Alcohol, and combinations thereof, 10 wt. %-20 wt. %, e.g., ca. 15 wt. % Medium-chain triglycerides, and 1 wt. %-3 wt. %, e.g., ca. 2 wt. %; Mono- and Di-glycerides; 0.05 wt. %-1 wt. %, e.g., ca. 0.2 wt. % of a viscosity modifying agent comprising Carbomer Homopolymer Type A; 0.5 wt. %-2 wt. %, e.g., ca. 1 wt. % of a water phase comprising Sodium Cetostearyl Sulfate and 25 wt. % to 75 wt. % Water; one or more preservatives comprising 0.5 wt. %-2 wt. %, ca. 1 wt. % benzyl alcohol; one or more buffers comprising 0.025 wt. %-0.2 wt. %, ca. 0.05 wt. % Citric Acid; and q.s. to pH 5-7.5 Sodium Hydroxide.

In some embodiments, the therapeutically active vehicle comprises an oil phase comprising 10 wt. %-15 wt. % fatty alcohols selected from Oleyl Alcohol, Cetyl Alcohol, Stearyl Alcohol, and combinations thereof, 15 wt. % Medium-chain triglycerides, and 1 wt. %-3 wt. %; Mono- and Di-glycerides; 0.05 wt. %-1 wt. %, of a viscosity modifying agent comprising Carbomer Homopolymer Type A; 0.5 wt. %-2 wt. % of a water phase comprising Sodium Cetostearyl Sulfate; 2 wt. %-8 wt. % propylene glycol; and 25 wt. % to 75 wt. % Water; one or more preservatives comprising 0.5 wt. %-2 wt. % benzyl alcohol; one or more buffers comprising 0.025 wt. %-0.2 wt. % Citric Acid; and q.s. to pH 5-7.5 Sodium Hydroxide.

In some embodiments, the therapeutically active vehicle comprises an oil phase comprising 10 wt. %-15 wt. % fatty alcohols selected from Oleyl Alcohol, Cetyl Alcohol, Stearyl Alcohol, and combinations thereof, 10 wt. %-20 wt. % Medium-chain triglycerides, and 2 wt. % Mono- and Di-glycerides; 0.05 wt. %-1 wt. % of a viscosity modifying agent comprising Carbomer Homopolymer Type A; 0.5 wt. %-2 wt. % of a water phase comprising Sodium Cetostearyl Sulfate, 2 wt. %-8 wt. % propylene glycol; and 25 wt. % to 75 wt. % Water; one or more preservatives comprising 0.5 wt. %-2 wt. % benzyl alcohol; one or more buffers comprising 0.025 wt. %-0.2 wt. % Citric Acid; and q.s. to pH 5-7.5 Sodium Hydroxide.

In some embodiments, the therapeutically active vehicle comprises an oil phase comprising 10 wt. %-15 wt. % fatty alcohols selected from Oleyl Alcohol, Cetyl Alcohol, Stearyl Alcohol, and combinations thereof, 10 wt. %-20 wt. % Medium-chain triglycerides, and 1 wt. %-3 wt. %; Mono- and Di-glycerides; 0.2 wt. % of a viscosity modifying agent comprising Carbomer Homopolymer Type A; 0.5 wt. %-2 wt. % of a water phase comprising Sodium Cetostearyl Sulfate, 2 wt. %-8 wt. % propylene glycol; and 25 wt. % to 75 wt. % Water; one or more preservatives comprising 0.5 wt. %-2 wt. % benzyl alcohol; one or more buffers comprising 0.025 wt. %-0.2 wt. % Citric Acid; and q.s. to pH 5-7.5 Sodium Hydroxide.

In some embodiments, the therapeutically active vehicle comprises an oil phase comprising 10 wt. %-15 wt. % fatty alcohols selected from Oleyl Alcohol, Cetyl Alcohol, Stearyl Alcohol, and combinations thereof, 10 wt. %-20 wt. % Medium-chain triglycerides, and 1 wt. %-3 wt. % Mono- and Di-glycerides; 0.05 wt. %-1 wt. % of a viscosity modifying agent comprising Carbomer Homopolymer Type A; 1 wt. % of a water phase comprising Sodium Cetostearyl Sulfate, 2 wt. %-8 wt. % propylene glycol; and 25 wt. % to 75 wt. % Water; one or more preservatives comprising 0.5 wt. %-2 wt. % benzyl alcohol; one or more buffers comprising 0.025 wt. %-0.2 wt. %, Citric Acid; and q.s. to pH 5-7.5 Sodium Hydroxide.

In some embodiments, the therapeutically active vehicle comprises an oil phase comprising 10 wt. %-15 wt. % fatty alcohols selected from Oleyl Alcohol, Cetyl Alcohol, Stearyl Alcohol, and combinations thereof, 10 wt. %-20 wt. % Medium-chain triglycerides, and 1 wt. %-3 wt. %; Mono- and Di-glycerides; 0.05 wt. %-1 wt. % of a viscosity modifying agent comprising Carbomer Homopolymer Type A; 0.5 wt. %-2 wt. % of a water phase comprising Sodium Cetostearyl Sulfate; 2 wt. %-8 wt. % propylene glycol; and 25 wt. % to 75 wt. % Water; one or more preservatives comprising 1 wt. % benzyl alcohol; one or more buffers comprising 0.025 wt. %-0.2 wt. % Citric Acid; and q.s. to pH 5-7.5 Sodium Hydroxide.

In some embodiments, the therapeutically active vehicle comprises an oil phase comprising 10 wt. %-15 wt. % fatty alcohols selected from Oleyl Alcohol, Cetyl Alcohol, Stearyl Alcohol, and combinations thereof, 10 wt. %-20 wt. % Medium-chain triglycerides, and 1 wt. %-3 wt. %; Mono- and Di-glycerides; 0.05 wt. %-1 wt. % of a viscosity modifying agent comprising Carbomer Homopolymer Type A; 0.5 wt. %-2 wt. % of a water phase comprising Sodium Cetostearyl Sulfate; 2 wt. %-8 wt. % propylene glycol; and 25 wt. % to 75 wt. % Water; one or more preservatives comprising 0.5 wt. %-2 wt. % benzyl alcohol; one or more buffers comprising 0.05 wt. % Citric Acid; and q.s. to pH 5-7.5 Sodium Hydroxide.

In some embodiments of the foregoing topical lotion, the additional active agent is disposed within the therapeutically active vehicle. In some embodiments, the additional active agent is disposed within the oil phase and the water phase of the therapeutically active vehicle. In some embodiments, the additional active agent is disposed within the oil phase of the therapeutically active vehicle. In some embodiments, the additional active agent is disposed within the water phase of the therapeutically active vehicle. In some embodiments, where the additional active agent is hydrophobic, it will be substantially disposed within the oil phase of the therapeutically active vehicle. In some embodiments, where the additional active agent is hydrophilic, it will be substantially disposed within the water phase of the therapeutically active vehicle. In some embodiments, where the additional active agent is amphiphilic, a portion of the additional active agent may be disposed within the oil phase and a portion of the additional active agent may be disposed within the water phase.

In an embodiment, the topical lotion comprises a therapeutically active vehicle and an additional active agent, wherein the therapeutically active vehicle provides an emulsion comprising:
an oil phase comprising:
   1.0 wt. % of an additional active agent;
   10 wt. % to 15 wt. % Fatty alcohols selected from Oleyl Alcohol, Cetyl Alcohol, Stearyl Alcohol, and combinations thereof;
   10 wt. % to 25 wt. % Medium-chain triglycerides; and
   1 wt. % to 3 wt. % Mono-glycerides and Di-glycerides;
a viscosity modifying agent comprising 0.05 wt. % to 1 wt. % Carbomer Homopolymer Type A;
a water phase comprising:
   0.5 wt. % to 2 wt. % Sodium Cetostearyl Sulfate; and 2 wt. % to 8 wt. % propylene glycol; and
   25 wt. % to 40 wt. % Water;
one or more preservatives comprising 0.5 wt. % to 2 wt. % benzyl alcohol;
one or more buffers comprising:
0.025 wt. % to 0.2 wt. % Citric Acid; and
Sodium Hydroxide, quantum satis (q.s.) to pH 5-7.5.

In an embodiment, the topical lotion comprises a therapeutically active vehicle and an additional active agent, wherein the therapeutically active vehicle provides an emulsion comprising:
an oil phase comprising:
   10 wt. % to 15 wt. % Fatty alcohols selected from Oleyl Alcohol, Cetyl Alcohol, Stearyl Alcohol, and combinations thereof;
   10 wt. % to 25 wt. % Medium-chain triglycerides; and
   1 wt. % to 3 wt. % Mono-glycerides and Di-glycerides;
a viscosity modifying agent comprising 0.05 wt. % to 1 wt. % Carbomer Homopolymer Type A;
a water phase comprising:
   1.0 wt. % of an additional active agent; 0.5 wt. % to 2 wt. % Sodium Cetostearyl Sulfate; and
   2 wt. % to 8 wt. % propylene glycol; and
   25 wt. % to 75 wt. % Water;
one or more preservatives comprising 0.5 wt. % to 2 wt. % benzyl alcohol;
one or more buffers comprising:
0.025 wt. % to 0.2 wt. % Citric Acid; and
Sodium Hydroxide, quantum satis (q.s.) to pH 5-7.5.

In some embodiments, a topical lotion comprises a therapeutically active vehicle and an additional active agent, wherein the therapeutically active vehicle provides an emulsion comprising:

| Component | Concentration (wt. %) w/w) |
|---|---|
| The additional active agent | 0.9-1.0, or 1.0-1.1 |
| Caprylic/Capric Triglycerides | 13-14, 14-15, 15-16, or 16-17 |
| Oleyl Alcohol | 8-9, 9-10, 10-11, or 11-12 |
| Sodium Cetostearyl Sulfate | 0.5-1.0, or 1.0-1.5 |
| Mono- and Di-glyceride | 12, or 2-3 |
| Carbopol 981 | 0.1-0.2, 0.2-0.3, 0.3-0.4, or 0.4-0.5 |
| (Carbomer Homopolymer Type A) | |
| Cetyl Alcohol | 0.5-0.8, 0.8-1.0, 1.0-1.2, or 1.2-1.5 |
| Stearyl Alcohol | 0.5-0.8, 0.8-1.0, 1.0-1.2, or 1.2-1.5 |
| Benzyl Alcohol | 0.5-0.8, 0.8-1.0, 1.0-1.2, or 1.2-1.5 |
| Propylene Glycol | 2-3, 3-4, 4-5, 5-6, 6-7, or 7-8 |
| Citric Acid, Anhydrous | 0.03-0.05, 0.05-0.07, or 0.07-0.1 |
| Sodium Hydroxide | q.s. pH 5-5.5, or 5.5-6 |
| Purified water | Balance |

In an embodiment, the topical lotion comprises a therapeutically active vehicle and an additional active agent, wherein the therapeutically active vehicle provides an emulsion comprising:

| Component | Concentration (wt. %) w/w) |
|---|---|
| The additional active agent | 0.9-1.1 |
| Caprylic/Capric Triglycerides | 13-17 |
| Oleyl Alcohol | 8-12 |
| Sodium Cetostearyl Sulfate | 0.5-1.5 |
| Mono- and Di-glyceride | 1-3 |
| Carbopol 981 | 0.1-0.5 |
| (Carbomer Homopolymer Type A) | |
| Cetyl Alcohol | 0.5-1.5 |
| Stearyl Alcohol | 0.5-1.5 |
| Benzyl Alcohol | 0.5-1.5 |
| Propylene Glycol | 2-8 |
| Citric Acid, Anhydrous | 0.03-0.1 |
| Sodium Hydroxide | q.s. pH 5-7.5 |
| Purified water | Balance |

In an embodiment, the topical lotion comprises a therapeutically active vehicle and an additional active agent, wherein the therapeutically active vehicle provides an emulsion comprising:

| | Concentration |
|---|---|
| Component | (wt. %) w/w) |
| The additional active agent | 1.0 |
| Caprylic/Capric Triglycerides | 15 |
| Oleyl Alcohol | 10 |
| Sodium Cetostearyl Sulfate | 1.0 |
| Mono- and Di-glyceride | 2.0 |
| Carbopol 981 | 0.2 |
| (Carbomer Homopolymer Type A) | |
| Cetyl Alcohol | 1.0 |
| Stearyl Alcohol | 1.0 |
| Benzyl Alcohol | 1.0 |
| Propylene Glycol | 5.0 |
| Citric Acid, Anhydrous | 0.05 |
| Sodium Hydroxide | q.s. pH 5.5 |
| Purified water | Balance |

In some embodiments of the foregoing topical lotion, the additional active agent may be selected from the group consisting of an AD agent, a natural moisturizing factor (NMF), a peptide/collagen synthesis agent, a skin rejuvenation agent, an acne agent, a skin protectant, an analgesic agent, an antipruritic agent, an antihistamine, a macrolide, and combinations thereof. In some embodiments, the additional active agent comprises a macrolide.

In some embodiments, an AD agent may be Ophiopogon japonicus (Mondo grass).

In some embodiments, an NMF may be selected from the group consisting of pyrrolidone carboxylic acid (PCA), hyaluronic acid, a ceramide, lactic acid, an amino acid, urea, squalene, and a combination thereof.

In some embodiments, a peptide/collagen synthesis agent may be selected from the group consisting of Palmitoyl Tripeptide-38, Palmitoyl Tripeptide-1, Palmitoyl Tetrapeptide-7, Acetyl hexapeptide 3, Acetyl hexapeptide 8, Acetyl hexapeptide 20, Palmitoyl oligopeptide, Lupine protein, a fractionated peptide extract (e.g., green peptides), and a combination thereof.

In some embodiments, the skin rejuvenation agent may be selected from the group consisting of a retinol, Retinyl Palmitate, Hydroxypinacolone Retinoate, Retinyl Lenoleate, bakuchiol extract (natural and green retinol alternative), Ceratonia siliqua (Locust bean gum), an Alpha Hydroxy Acid, a Beta Hydroxy Acid, and a combination thereof.

In some embodiments, the acne agent may be selected from the group consisting of Punica granatum Pericarp Extract (pomegranate peel extract), Tea tree oil, Salicylic acid, benzoyl peroxide (BPO), sulfur, and a combination thereof.

In some embodiments, the skin protectant may be selected from the group consisting of allantoin, calamine, and a combination thereof.

In some embodiments, the analgesic agent may be selected from the group consisting of lidocaine, pramoxine, benzocaine, hydrocortisone, and a combination thereof.

In some embodiments, the antipruritic agent may be selected from the group consisting of mint oil, menthol, camphor, Hydroxyphenyl Propamidobenzoic Acid, Avena sativa (Oat) Kernel Extract, Beta Glucan, an Avenanthramide, and combinations thereof.

In some embodiments, the antihistamine may be diphenylhydramine.

In some embodiments, the macrolide may be selected from the group consisting of rapamycin, pimecrolimus, temsirolimus, everolimus, and tacrolimus. In some embodiments, the additional active agent may be pimecrolimus.

In an embodiment of the foregoing topical lotion, the topical lotion comprises a therapeutically active vehicle and an additional active agent, wherein the additional active agent is pimecrolimus, and wherein the therapeutically active vehicle provides an emulsion comprising:

An oil phase comprising:
1.0 wt. % Pimecrolimus;
   10 wt. % to 15 wt. % Fatty alcohols selected from Oleyl Alcohol, Cetyl Alcohol, Stearyl Alcohol, and combinations thereof;
   10 wt. % to 20 wt. % Medium-chain triglycerides; and
   1 wt. % to 3 wt. % Mono-glycerides and Di-glycerides;
a viscosity modifying agent comprising 0.05 wt. % to 1 wt. % Carbomer Homopolymer Type A;
a water phase comprising:
   0.5 wt. % to 2 wt. % Sodium Cetostearyl Sulfate; and
   2 wt. % to 8 wt. % propylene glycol; and
   25 wt. % to 40 wt. % Water;
one or more preservatives comprising 0.5 wt. % to 2 wt. % benzyl alcohol;
one or more buffers comprising:
   0.025 wt. % to 0.2 wt. % Citric Acid; and
Sodium Hydroxide, quantum satis (q.s.) to pH 5-7.5.

In an embodiment, the topical lotion comprises a therapeutically active vehicle and an additional active agent, wherein the additional active agent is pimecrolimus, and wherein the therapeutically active vehicle provides an emulsion comprising:

| | Concentration |
|---|---|
| Component | (wt. %) w/w) |
| Pimecrolimus | 0.9-1.1 |
| Caprylic/Capric Triglycerides | 13-17 |
| Oleyl Alcohol | 8-12 |
| Sodium Cetostearyl Sulfate | 0.5-1.5 |
| Mono- and Di-glyceride | 1-3 |
| Carbopol 981 | 0.1-0.5 |
| (Carbomer Homopolymer Type A) | |
| Cetyl Alcohol | 0.5-1.5 |
| Stearyl Alcohol | 0.5-1.5 |
| Benzyl Alcohol | 0.5-1.5 |
| Propylene Glycol | 2-8 |
| Citric Acid, Anhydrous | 0.03-0.1 |
| Sodium Hydroxide | q.s. pH 5-7.5 |
| Purified water | Balance |

In an embodiment, the topical lotion comprises a therapeutically active vehicle and an additional active agent, wherein the additional active agent is pimecrolimus, and wherein the therapeutically active vehicle provides an emulsion comprising:

| Component | Concentration (wt. %) w/w) |
|---|---|
| Pimecrolimus | 0.9-1.0, or 1.0-1.1 |
| Caprylic/Capric | 13-14, 14-15, 15-16, or 16-17 |
| Triglycerides | |
| Oleyl Alcohol | 8-9, 9-10, 10-11, or 11-12 |
| Sodium Cetostearyl Sulfate | 0.5-1.0, or 1.0-1.5 |
| Mono- and Di-glyceride | 12, or 2-3 |
| Carbopol 981 | 0.1-0.2, 0.2-0.3, 0.3-0.4, or 0.4-0.5 |
| (Carbomer Homopolymer | |
| Type A) | |
| Cetyl Alcohol | 0.5-0.8, 0.8-1.0, 1.0-1.2, or 1.2-1.5 |
| Stearyl Alcohol | 0.5-0.8, 0.8-1.0, 1.0-1.2, or 1.2-1.5 |
| Benzyl Alcohol | 0.5-0.8, 0.8-1.0, 1.0-1.2, or 1.2-1.5 |
| Propylene Glycol | 2-3, 3-4, 4-5, 5-6, 6-7, or 7-8 |
| Citric Acid, Anhydrous | 0.03-0.05, 0.05-0.07, or 0.07-0.1 |
| Sodium Hydroxide | q.s. pH 5-5.5, or 5.5-6 |
| Purified water | Balance |

In an embodiment, the topical lotion comprises a therapeutically active vehicle and an additional active agent, wherein the additional active agent is pimecrolimus, and wherein the therapeutically active vehicle provides an emulsion comprising:

| | Concentration |
|---|---|
| Component | (wt. %) w/w) |
| Pimecrolimus | 1.0 |
| Caprylic/Capric Triglycerides | 15 |
| Oleyl Alcohol | 10 |
| Sodium Cetostearyl Sulfate | 1.0 |
| Mono- and Di-glyceride | 2.0 |
| Carbopol 981 | 0.2 |
| (Carbomer Homopolymer Type A) | |
| Cetyl Alcohol | 1.0 |
| Stearyl Alcohol | 1.0 |
| Benzyl Alcohol | 1.0 |
| Propylene Glycol | 5.0 |
| Citric Acid, Anhydrous | 0.05 |
| Sodium Hydroxide | q.s. pH 5.5 |
| Purified water | Balance |

Pimecrolimus is a 33-epi-chloro derivative of ascomycin, having the structure:

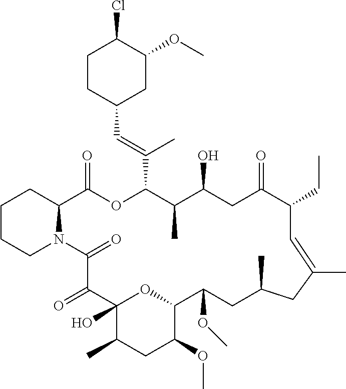 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](Cl)[C@H](OC)C3)[C@H](C)[C@@H](O)CC1=O)[C@H](C)C[C@@H]2OC]

The exact mechanism of pimecrolimus is not known with certainty, but it is a calcineurin inhibitor, and appears to have immunosuppressive and anti-inflammatory effects due to its inhibition of various cytokine pathways. Pimecrolimus has proved difficult to formulate, due to its relative insolubility.

In an embodiment, the invention includes a method of treating a dermatological condition with a topical lotion described herein. In some embodiments, the dermatological condition is AD. In some embodiments, the dermatological condition is moderate to severe AD.

In some embodiments wherein the dermatological condition is AD, and more particularly moderate to severe AD, patients may be evaluated and treatment success may be assessed using methods customary in the relevant art.

In some embodiments wherein the dermatological disease is AD, and more particularly moderate to severe AD, patient evaluation and treatment success may be assessed, e.g., using a 5-point Investigator Global Assessment (IGA) scale from clear to severe; the Eczema Area Severity Index (EASI); the total affected body surface area (TBSA); assessment of pruritus (itching) and sleep disturbance; Patient-Oriented Eczema Measure (POEM) from subjects and parents/legal guardians of pediatric subjects; and investigator's assessment of signs and symptoms of AD.

IGA may be assessed, e.g., on a static scale ranging from 0 (clear) to 4 (severe):

| Score | Grade | Description |
|---|---|---|
| 0 | Clear | No inflammatory signs of AD (no |
| | | erythema, no induration/papulation, |
| | | no lichenification, no oozing/ |
| | | crusting). Post-inflammatory |
| | | hyperpigmentation and/or |
| | | hypopigmentation may be present. |
| 1 | Almost | Barely perceptible erythema, barely |
| | clear | perceptible induration/papulation, and/or |
| | | minimal lichenification. |
| | | No oozing or crusting. |
| 2 | Mild | Slight but definite erythema (pink), slight |
| | | but definite induration/papulation, |
| | | and/or slight but definite lichenification. |
| | | No oozing or crusting. |
| 3 | Moderate | Clearly perceptible erythema |
| | | (dull red), clearly perceptible |
| | | induration/papulation, and/or clearly |
| | | perceptible lichenification. Oozing |
| | | and crusting may be present. |
| 4 | Severe | Marked erythema (deep or bright red), |
| | | marked induration/papulation, |
| | | and/or marked lichenification. Disease |
| | | is wide-spread in extent. Oozing |
| | | or crusting may be present. |

EASI is a composite score based on the evaluated severity of 4 key signs of AD (i.e., erythema, infiltration/papulation, excoriation and lichenification), and the extent of disease in each of the 4 body regions (i.e., head/neck, trunk, upper limbs, and lower limbs). The area of involvement (affected by inflammation, not including dry skin) of each of the 4 body regions is determined and represented by a numeric coded value based on a scale from 0 to 6. The 4 body regions are assessed separately for each sign/symptom, and the average degree of severity of each sign in each of the 4 body parts is assigned a score of 0 (none) to 3 (severe) on a scale that allows half-unit increments. The EASI score ranges from 0 to 72, with higher scores being worse.

The area of involvement (affected by inflammation, not including dry skin) of each of the 4 body regions is determined and represented by a numeric coded value based on a scale from 0 to 6:

| Score | Area of Involvement |
|---|---|
| 0 | No eruption |
| 1 | <10% |
| 2 | 10%-29% |
| 3 | 30%-49% |
| 4 | 50%-69% |
| 5 | 70%-89% |
| 6 | 90%-100% |

The four body regions are assessed separately for each sign/symptom, and the average degree of severity of each sign in each of the 4 body parts is assigned a score of 0 (none) to 3 (severe) on a scale that allows half-unit increments:

| Sign/symptom | Score | Grade | Description | |
|---|---|---|---|---|
| Erythema (E) | 0 | None | No erythema present | |
| | 1 | Mild | Mild erythema, very | |
| | | | light pink | |
| | 2 | Moderate | Pink red clearly | |
| | | | distinguishable | |
| | | | perceptible erythema | |
| | 3 | Severe | Bright red | |
| Infiltration/ | 0 | None | None | |
| Papulation | | | | |
| (1) | | | | |
| | 1 | Mild | Barely perceptible | |
| | | | elevation | |
| | 2 | Moderate | Clearly perceptible | |
| | | | elevation, but not | |
| | | | extensive | |
| | 3 | Severe | Marked and | |
| | | | extensive elevation | |
| Excoriation (X) | 0 | None | None | |
| | 1 | Mild | Scant evidence of | |
| | | | excoriations with no | |
| | | | signs of deeper skin | |
| | | | damage (erosion, | |
| | | | crust) | |
| | 2 | Moderate | Severe linear marks | |
| | | | of skin with some | |
| | | | showing evidence of | |
| | | | deeper skin injury | |
| | | | (erosion, crust) | |
| | 3 | Severe | Many erosive or | |
| | | | crusty lesions | |
| Lichenification (L) | 0 | None | None | |
| | 1 | Mild | Slight thickening of | |
| | | | the skin discernible | |
| | | | only by touch and | |
| | | | with skin markings | |
| | | | minimally | |
| | | | exaggerated | |
| | 2 | Moderate | Definite thickening | |
| | | | of the skin with skin | |
| | | | marking exaggerated | |
| | | | so that they form a | |
| | | | visible crisscross | |
| | | | pattern | |
| | 3 | Severe | Thickened indurated | |
| | | | skin with skin | |
| | | | markings visibly | |
| | | | portraying an | |
| | | | exaggerated | |
| | | | crisscross pattern | |
| | Head/neck | Upper limbs | Trunk | Lower limbs |
| ≥8 years | 0.1 | 0.2 | 0.3 | 0.4 |
| <8 years | 0.2 | 0.2 | 0.3 | 0.3 |

E = erythema;
I = infiltration/papulation;
EX = excoriation;
L = lichenification.
Area is defined on a 7-point scale, where 0 = no eruption, 1 = <10%, 2 = 10%-29%, 3 = 30%-49%, 4 = 50%-69%, 5 = 70%-89%, 6 = ≥90%.
*A value is assigned to P based on subject age as shown below:

An EASI score of 0 indicates clear or no eczema, 0.1 to 1.0 indicates almost clear, 1.1 to 7 indicates mild disease, 7.1 to 21 indicates moderate disease, 21.1 to 50 indicates severe disease, and greater than 51 indicates very severe disease. EASI 75 is defined as at least a 75% reduction in the Eczema Area Severity Index (EASI) score from baseline.

Pruritis and sleep disturbance may be assessed on the following scale, by the subject or the subject's parent or guardian:

| | | |
|---|---|---|
| Score | Grade | Description |

Pruritus

| | | |
|---|---|---|
| 0 | None | None |
| 1 | Mild | Occasional, slight itching/scratching |
| 2 | Moderate | Constant or intermittent itching/scratching/ |
| | | discomfort which is not disturbing sleep |
| 3 | Severe | Bothersome itching/scratching/discomfort |
| | | which is disturbing sleep |

Sleep Disturbance

| | | |
|---|---|---|
| 0 | None | None |
| 1 | Mild | Occasional, few disturbances |
| 2 | Moderate | Intermittent disturbances |
| 3 | Severe | Bothersome and disruptive disturbances |

The subject or parent or legal guardian may also assess how quickly relief of pruritis is achieved following application of the formulation, e.g., 1-2 days post first application, 3-4 days post first application, or 5-6 days post first application.

Other signs and symptoms of AD to be assessed by the investigator may include hyperpigmentation, hypopigmentation, and dry skin, e.g., on a 4-point scale:

| | | |
|---|---|---|
| Score | Grade | Description |

Hypopigmentation

| | | |
|---|---|---|
| 0 | None | No evidence |
| 1 | Mild | Slight, barely perceptible |
| 2 | Moderate | Definite, evident |
| 3 | Severe | Marked, prominent |

Hyperpigmentation

| | | |
|---|---|---|
| 0 | None | No evidence |
| 1 | Mild | Slight, barely perceptible |
| 2 | Moderate | Definite, evident |
| 3 | Severe | Marked, prominent |

Dry Skin

| | | |
|---|---|---|
| 0 | None | No dryness |
| 1 | Mild | Slight, but definite roughness |
| 2 | Moderate | Definite roughness |
| 3 | Severe | Marked roughness |

The Patient Oriented Eczema Measure (POEM) is a self-assessment using a tool available from the University of Nottingham (see https://www.nottingham.ac.uk/research/groups/cebd/resources/poem.aspx).

In some embodiments of the methods described herein, the topical lotion is applied twice daily.

In some embodiments of the methods described herein, the duration of treatment is greater than four weeks.

In some embodiments of the methods described herein, the duration of treatment is about six weeks.

In some embodiments of the methods described herein, the subject at the start of treatment exhibits moderate to severe AD, characterized by (a) clearly perceptible erythema (dull red), clearly perceptible induration/papulation, and/or clearly perceptible lichenification, wherein oozing or crusting may be present; or (b) marked erythema (deep or bright red), marked induration/papulation, and/or marked lichenification, wherein the disease is wide-spread in extent, and wherein oozing or crusting may be present.

In some embodiments of the methods described herein, the subject at the start of treatment exhibits severe AD, characterized by marked erythema (deep or bright red), marked induration/papulation, and/or marked lichenification, wherein the disease is wide-spread in extent, and wherein oozing or crusting may be present.

In some embodiments of the methods described herein, the subject at the start of treatment has an Investigator Global Assessment (IGA) Scale score of greater than 3.

In some embodiments of the methods described herein, the subject at the start of treatment has an EASI score of greater than 21.

In some embodiments of the methods described herein, the subject exhibits at least a 2-grade reduction in the Investigator Global Assessment (IGA) Scale and exhibits an IGA that equates to “Clear” (0) or “Almost Clear” (1) after at least four weeks of treatment, e.g., after six weeks of treatment.

In some embodiments of the methods described herein, the subject exhibits 75% reduction in the Eczema Area Severity Index (EASI) score after at least four weeks of treatment, e.g., after six weeks of treatment.

In some embodiments of the methods described herein, the subject exhibits at least a 1 grade improvement from baseline in the severity of pruritus score and a pruritus score equating to “none” or “mild” after at least one week of treatment, e.g., after two weeks of treatment.

In some embodiments of the methods described herein, the subject is at least two years old.

In some embodiments of the methods described herein, in a clinical trial after six weeks of treatment the percentage of participants who have at least a 2-grade reduction from baseline in the Investigator Global Assessment (IGA) Scale and have an IGA that equated to “Clear” or “Almost Clear” is at least 40%, e.g., at least 42%.

In some embodiments of the methods described herein, in a clinical trial after six weeks of treatment the percentage of participants who reach EASI 75 (defined as at least a 75% reduction in the Eczema Area Severity Index (EASI) score from the start of treatment) is at least 45%.

Unless stated otherwise, all percentages of composition components given in this specification are by weight based on a total composition or formulation weight of 100%.

The compositions and formulations as provided herein are described and claimed with reference to their ingredients, as is usual in the art. As would be evident to one skilled in the art, the ingredients may in some instances react with one another, so that the true composition of the final formulation may not correspond exactly to the ingredients listed. Thus, it should be understood that the invention extends to the product of the combination of the listed ingredients.

It will be understood that in certain formulations a particular ingredient may have more than one function. For example, relatively lipophilic nonpolar surfactants such as glycerol monostearate or glycerol distearate may form all or part of the oil phase of an emulsion formulation, as well as helping to keep the oil phase emulsified and stable.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

While certain embodiments of the present invention have been described and/or exemplified above, various other embodiments will be apparent to those skilled in the art from the foregoing disclosure. The invention is, therefore, not limited to the particular embodiments described and/or exemplified, but is capable of considerable variation and modification without departure from the scope and spirit of the appended claims.

### EXAMPLES

The following examples describe the invention in further detail. These examples are provided for illustrative purposes only, and should in no way be considered as limiting the invention.

### Example 1: Exemplary IDP-124 Topical Vehicle Lotion

An exemplary topical lotion comprising a therapeutically active vehicle is set forth in Table 1.

TABLE 1
Quantitative Composition of Topical Vehicle Lotion

| | | Concentration |
|---|---|---|
| | | (wt. %) |
| Component | Function | (w/w) |
| Caprylic/Capric | Solvent | 15 |
| Triglycerides | | |
| Oleyl Alcohol | Solvent | 10 |
| Sodium Cetostearyl Sulfate | Anionic Emulsifier | 1 |
| Mono- and Di-glycerides | Consistency factor, co- | 2 |
| | emulsifier | |
| Carbopol 981 | Viscosity modifying | 0.2 |
| (Carbomer Homopolymer | agent | |
| Type A) | | |
| Cetyl Alcohol | Consistency factor, co- | 1 |
| | emulsifier | |
| Stearyl Alcohol | Consistency factor, co- | 1 |
| | emulsifier | |
| Benzyl Alcohol | Preservative | 1 |
| Propylene Glycol | Consistency factor | 5 |
| Citric Acid, Anhydrous | Buffer | 0.05 |
| Sodium Hydroxide | Buffer | q.s. pH 5-7.5 |
| Purified water | | q.s. to 100% |

The exemplary formulation set forth in Table 1 is prepared using the following method.

### Preparation of the Aqueous Phase:

1. Add purified water into a low shear convection mixer with high shear capabilities. Initiate mixing, heating, and homogenization and then add sodium cetostearyl sulfate.
2. Stop mixing and homogenizing, and hold until Step 8.

### Preparation of the Oil Phase:

3. Add oleyl alcohol into a jacketed mixing tank and initiate mixing.
4. Add medium-chain triglycerides, mono- and di-glycerides, cetyl alcohol, and stearyl alcohol, and then initiate heating. Visually confirm complete dissolution.
5. Maintain mixing and hold until Step 9.

### Preparation of Carbomer Dispersion:

6. Add purified water and Carbopol-981 into a Mixing Tank using a Tri-Blender and mix the contents. Visually confirm a lump-free dispersion.
7. Add propylene glycol and benzyl alcohol and continue mixing.

### Preparation of the Topical Pimecrolimus Lotion:

8. Confirm that the Aqueous Phase batch temperature is ≥80° C.
9. Confirm that the Oil Phase batch temperature is ≥80° C.
10. Add the Oil Phase into the low shear convection mixer with high shear capabilities (containing the Aqueous Phase) and mix and homogenize. Slightly cool the mixture.
11. Add the Carbomer Dispersion and continue to mix and homogenize.
12. Add the Buffer Solution (a pre-made solution of purified water, citric acid anhydrous, and sodium hydroxide pellets) and continue to mix and homogenize.
13. Cool the mixture and continue to mix and homogenize.
14. Ensure that pH is 5.5 to 6.0. If pH is below 5.5, add a pre-made solution of purified water and sodium hydroxide pellets until the pH is between 5.5 and 6.0.

### Example 2: Exemplary IDP-124 Pimecrolimus Topical Lotion

An exemplary topical lotion comprising a therapeutically active vehicle and an additional active agent (i.e., pimecrolimus) is set forth in Table 2.

TABLE 2
Quantitative Composition of Topical Pimecrolimus Lotion

| | | Concentration |
|---|---|---|
| | | (wt. %) |
| Component | Function | (w/w) |
| Pimecrolimus | Drug Substance | 1.0 |
| Caprylic/Capric | Solvent | 15 |
| Triglycerides | | |
| Oleyl Alcohol | Solvent | 10 |
| Sodium Cetostearyl Sulfate | Anionic Emulsifier | 1 |
| Mono- and Di-glycerides | Consistency factor, co- | 2 |
| | emulsifier | |
| Carbopol 981 | Viscosity modifying | 10.2 |
| (Carbomer Homopolymer | agent | |
| Type A) | | |
| Cetyl Alcohol | Consistency factor, co- | 1 |
| | emulsifier | |
| Stearyl Alcohol | Consistency factor, co- | 1 |
| | emulsifier | |
| Benzyl Alcohol | Preservative | 1 |
| Propylene Glycol | Consistency factor | 5 |
| Citric Acid, Anhydrous | Buffer | 0.05 |
| Sodium Hydroxide | Buffer | q.s. pH 5-6 |
| Purified water | | q.s. to 100% |

The exemplary formulation set forth in Table 2 is prepared using the following method.

### Preparation of the Aqueous Phase:

1. Add purified water into a low shear convection mixer with high shear capabilities. Initiate mixing, heating, and homogenization and then add sodium cetostearyl sulfate.
2. Stop mixing and homogenizing, and hold until Step 8.

### Preparation of the Oil Phase:

3. Add oleyl alcohol into a jacketed mixing tank and initiate mixing.
4. Add medium-chain triglycerides, mono- and di-glycerides, cetyl alcohol, stearyl alcohol, and pimecrolimus, and then initiate heating. Visually confirm complete dissolution.
5. Maintain mixing and hold until Step 9.

### Preparation of Carbomer Dispersion:

6. Add purified water and Carbopol-981 into a Mixing Tank using a Tri-Blender and mix the contents. Visually confirm a lump-free dispersion.
7. Add propylene glycol and benzyl alcohol and continue mixing.

### Preparation of the Topical Lotion:

8. Confirm that the Aqueous Phase batch temperature is ≥80° C.
9. Confirm that the Oil Phase batch temperature is ≥80° C.
10. Add the Oil Phase into the low shear convection mixer with high shear capabilities (containing the Aqueous Phase) and mix and homogenize. Slightly cool the mixture.
11. Add the Carbomer Dispersion and continue to mix and homogenize.
12. Add the Buffer Solution (a pre-made solution of purified water, citric acid anhydrous, and sodium hydroxide pellets) and continue to mix and homogenize.
13. Cool the mixture and continue to mix and homogenize.
14. Ensure that pH is 5.5 to 6.0. If pH is below 5.5, add a pre-made solution of purified water and sodium hydroxide pellets until the pH is between 5.5 and 6.0.

### Example 3: Treatment of Moderate to Severe AD

The Topical Vehicle Lotion of Example 1 and Topical Pimecrolimus Lotion of Example 2 were clinically tested to treat children (over the age of 2) and adults suffering from moderate to severe AD using the following protocols.

In a first trial, approximately 348 subjects, ages 2 and older, are randomized into two groups: 232 subjects to IDP-124 lotion (the lotion of Example 2, applied twice daily) and 116 subjects to IDP-124 Vehicle Lotion (the lotion of Example 1, applied twice daily). The study consists of a 6-week double-blind treatment period and 4-week, post-treatment follow-up. Study visits include: Screening, Baseline, and Weeks 1, 2, 4, 6, and 10 (subjects return to the clinic at Week 10 for a post-treatment follow-up visit). A second trial with substantially the same protocol is carried out, with 326 subjects (216 subjects in the test arm, and 110 subjects in the vehicle arm).

The key inclusion and exclusion criteria are as follows:

### Key Inclusion Criteria:

Male or female at least 2 years of age and older
Written and verbal informed consent must be obtained; subjects less than age of consent must sign an assent for the study and a parent or a legal guardian must sign the informed consent (if subject reaches age of consent during the study they should be re-consented at the next study visit)·
Nonimmunocompromised male or female who failed to respond adequately to other topical prescription treatment for AD or for whom those treatments are not advisable
Subjects must be willing to comply with study instructions and return to the clinic for required visits; subjects under the age of consent must be accompanied by the parent or legal guardian at the time of assent/consent signing

### Key Exclusion Criteria:

Females who are pregnant, breast feeding, or who wish to become pregnant during the study period
Active cutaneous bacterial or viral infection in any treatment area at Baseline (e.g., clinically infected AD)
Sunburn, extensive scarring, or pigmented lesion(s) in any treatment area at Baseline, which would interfere with evaluations
History of confounding skin conditions, e.g., psoriasis, rosacea, erythroderma, or ichthyosis (other than ichthyosis vulgaris)

The percentage of participants who have at least a 2-grade reduction from baseline in the Investigator Global Assessment (IGA) Scale and have an IGA that equated to “Clear” or “Almost Clear,” from baseline to Week 10, is determined. Evaluations are scored using a 5-point scale from clear (0) to severe (4), with higher numbers indicating worse scores:

| | | Control | | Control |
|---|---|---|---|---|
| | Pimecrolimus | Vehicle | Pimecrolimus | Vehicle |
| | Lotion | Lotion | Lotion | Lotion |
| | (Trial 1) | (Trial 1) | (Trial 2) | (Trial 2) |
| Overall Number of | 224 | 113 | 216 | 110 |
| Participants | | | | |
| Analyzed | | | | |
| Week 2 | 9.82% | 5.31% | 18.89% | 8.73% |
| Week 4 | 26.25% | 16.46% | 28.70% | 24.91% |
| Week 6 | 42.95% | 27.96% | 42.04% | 32.73% |
| [primary endpoint] | | | | |
| Week 10 | 35.71% | 24.96% | 31.67% | 29.64% |

The percentage of patients who reach EASI 75 (defined as at least a 75% reduction in the Eczema Area Severity Index (EASI) score from Baseline) is assessed:

| | | Control | | Control |
|---|---|---|---|---|
| | Pimecrolimus | Vehicle | Pimecrolimus | Vehicle |
| | Lotion | Lotion | Lotion | Lotion |
| | (Trial 1) | (Trial 1) | (Trial 2) | (Trial 2) |
| Overall Number of | 224 | 113 | 216 | 110 |
| Participants | | | | |
| Analyzed | | | | |
| Week 4 | 31.79% | 26.55% | 33.61% | 26.18% |
| Week 6 | 46.61% | 37.35% | 46.11% | 37.45% |
| Week 10 | 42.68% | 33.10% | 43.24% | 34.00% |

The percentage of participants who have at least a 1 grade improvement from baseline in the severity of pruritus score and a pruritus score equating to “none” or “mild” is assessed:

| | | Control | | Control |
|---|---|---|---|---|
| | Pimecrolimus | Vehicle | Pimecrolimus | Vehicle |
| | Lotion | Lotion | Lotion | Lotion |
| | (Trial 1) | (Trial 1) | (Trial 2) | (Trial 2) |
| Overall Number of | 224 | 113 | 216 | 110 |
| Participants | | | | |
| Analyzed | | | | |
| Week 1 | 41.20% | 34.31% | 46.19% | 37.41% |
| Week 2 | 51.34% | 43.30% | 52.76% | 39.81% |

As demonstrated herein, while the Topical Pimecrolimus Lotion (Example 2) showed greater activity in the foregoing studies than the Topical Vehicle Lotion (Example 1), both Topical Lotions demonstrated independent activity for the treatment of moderate to severe AD.

### Example 4: Hydration Study for IDP-124 Topical Vehicle Lotion

The Topical Vehicle Lotion of Example 1 was tested in a study with human subjects. The lotion was applied to subject forearms and then the moisture level was measured via a moisture meter apparatus to determine changes in skin moisture at three time periods. This study was to confirm the applicability of the Topical Vehicle Lotion of Example 1 as a moisturizer and having moisturizing effect on human skin.

The testing parameters for the study were as follows. Subjects were asked to wash forearms with soap and water 30 minutes prior to the evaluation of the untreated spot (baseline), and the treated spot with the test article (i.e., Topical Vehicle Lotion of Example 1). At time zero, three (3) readings from the untreated spot were taken, and the average reading was calculated. At time zero, the test article was applied to the designated treated spot on the forearm, and three (3) readings were taken, and the average reading was calculated. Then, the procedure was repeated at one (1) hour, and two (2) hour time points for both untreated, and the treated spot with the test article.

The result of the study is as follows with the percent increase in moisture of the treated spot (with Topical Vehicle Lotion of Example 1) as compared to the untreated spot (base line):

| Time Point | Zero | One Hour | Two Hours |
|---|---|---|---|
| Percent Increase in | 261.71% | 98.43% | 74.05% |
| Moisture form the | | | |
| Baseline (untreated | | | |
| spot) | | | |

As compared to baseline, the foregoing study demonstrates that the IDP-124 Vehicle Lotion (the Topical Vehicle Lotion of Example 1) provides moisturizing effect through three hours following application to human skin.

While the disclosure has been described with respect to specific examples including presently preferred modes of carrying out the disclosure, those skilled in the art will appreciate that there are numerous variations and permutations of the above-described methods and formulations. It is to be understood that other embodiments may be utilized and structural and functional modifications may be made without departing from the scope of the present disclosure. Thus, the scope of the disclosure should be construed broadly as set forth in the appended claims.

1. A method for treating atopic dermatitis (AD) in a subject in need thereof, comprising administering to the affected area a topical pharmaceutical lotion comprising a therapeutically active vehicle, wherein the therapeutically active vehicle comprises:
an oil phase comprising:
10 wt. % to 15 wt. % Fatty alcohols selected from Oleyl Alcohol, Cetyl Alcohol, Stearyl Alcohol, and combinations thereof;
10 wt. % to 20 wt. % Medium-chain triglycerides; and
1 wt. % to 3 wt. % Mono-glycerides and Di-glycerides;
a viscosity modifying agent comprising 0.05 wt. % to 1 wt. % Carbomer Homopolymer Type A;
a water phase comprising:
0.5 wt. % to 2 wt. % Sodium Cetostearyl Sulfate; and
25 wt. % to 75 wt. % Water;
one or more preservatives comprising 0.5 wt. % to 2 wt. % benzyl alcohol;
one or more buffers comprising:
0.025 wt. % to 0.2 wt. % Citric Acid; and
Sodium Hydroxide, quantum satis (q.s.) to pH 5-7.5.

2. The method of claim 1, wherein the water phase further comprises 2 wt. % to 8 wt. % propylene glycol.

3. The method of claim 1 or 2, wherein the oil phase comprises 15 wt. % Medium-chain triglycerides.

4. The method of any foregoing claim, wherein the oil phase comprises 2 wt. % Mono-glycerides and Di-glycerides.

5. The method of any foregoing claim, wherein the viscosity modifying agent comprises 0.2 wt. % Carbomer Homopolymer Type A.

6. The method of any foregoing claim, wherein the water phase comprises 1 wt. % Sodium Cetostearyl Sulfate.

7. The method of any foregoing claim, wherein the one or more preservatives comprise 1 wt. % benzyl alcohol.

8. The method of any foregoing claim, wherein the one or more buffers comprise 0.05 wt. % citric acid.

9. The method of any foregoing claim, wherein the Sodium Hydroxide is q.s. to pH 5.5.

10. The method of any foregoing claim, wherein the AD is moderate to severe AD.

11. The method of any foregoing claim wherein the topical pharmaceutical lotion is an emulsion.

12. The method of any foregoing claim wherein the topical pharmaceutical lotion is administered to the affected area twice daily.

13. The method of any foregoing claim wherein the duration of treatment is at least four weeks.

14. The method of any foregoing claim wherein the duration of treatment is six weeks.

15. The method of any foregoing claim wherein the atopic dermatitis is severe atopic dermatitis.

16. The method of any foregoing claim wherein the subject at the start of treatment exhibits moderate to severe AD, characterized by:
(a) clearly perceptible erythema (dull red), clearly perceptible induration/papulation, and/or clearly perceptible lichenification, wherein oozing or crusting may be present; or
(b) marked erythema (deep or bright red), marked induration/papulation, and/or marked lichenification, wherein the disease is wide-spread in extent, and wherein oozing or crusting may be present.

17. The method of any foregoing claim wherein the subject at the start of treatment exhibits severe AD, characterized by marked erythema (deep or bright red), marked induration/papulation, and/or marked lichenification, wherein the disease is wide-spread in extent, and wherein oozing or crusting may be present.

18. The method of any foregoing claim wherein the subject at the start of treatment has an Investigator Global Assessment (IGA) Scale score of greater than 3.

19. The method of any foregoing claim wherein the subject at the start of treatment has an EASI score of greater than 21.

20. The method of any foregoing claim wherein the subject exhibits at least a 2-grade reduction in the Investigator Global Assessment (IGA) Scale and exhibits an IGA that equates to “Clear” (0) or “Almost Clear” (1) after at least four weeks of treatment, e.g., after six weeks of treatment.

21. The method of any foregoing claim wherein the subject exhibits 75% reduction in the Eczema Area Severity Index (EASI) score after at least four weeks of treatment, e.g., after six weeks of treatment.

22. The method of any foregoing claim wherein the subject exhibits at least a 1 grade improvement from baseline in the severity of pruritus score and a pruritus score equating to “none” or “mild” after at least one week of treatment, e.g., after two weeks of treatment.

23. The method of any foregoing claim wherein the topical pharmaceutical lotion comprises an additional active agent.

24. The method of claim 23, wherein the topical pharmaceutical lotion comprises about 1-1.5 wt. % of the additional active agent.

25. The method of claim 23 or 24, wherein the additional active agent is selected from the group consisting of an AD agent, a natural moisturizing factor (NMF), a peptide/collagen synthesis agent, a skin rejuvenation agent, an acne agent, a skin protectant, an analgesic agent, an antipruritic agent, an antihistamine, a macrolide, and combinations thereof.

26. The method of claim 25, wherein the AD agent is Ophiopogon japonicus (Mondo grass).

27. The method of claim 25, wherein the NMF may be selected from the group consisting of pyrrolidone carboxylic acid (PCA), hyaluronic acid, a ceramide, lactic acid, an amino acid, urea, squalene, and a combination thereof.

28. The method of claim 25, wherein the peptide/collagen synthesis agent is selected from the group consisting of Palmitoyl Tripeptide-38, Palmitoyl Tripeptide-1, Palmitoyl Tetrapeptide-7, Acetyl hexapeptide 3, Acetyl hexapeptide 8, Acetyl hexapeptide 20, Palmitoyl oligopeptide, Lupine protein, a fractionated peptide extract (e.g., green peptides), and a combination thereof.

29. The method of claim 25, wherein the skin rejuvenation agent is selected from the group consisting of a retinol, Retinyl Palmitate, Hydroxypinacolone Retinoate, Retinyl Lenoleate, bakuchiol extract (natural and green retinol alternative), Ceratonia siliqua (Locust bean gum), an Alpha Hydroxy Acid, a Beta Hydroxy Acid, and a combination thereof.

30. The method of claim 25, wherein the acne agent is selected from the group consisting of Punica granatum Pericarp Extract (pomegranate peel extract), Tea tree oil, Salicylic acid, benzoyl peroxide (BPO), sulfur, and a combination thereof.

31. The method of claim 25, wherein the skin protectant is selected from the group consisting of allantoin, calamine, and a combination thereof.

32. The method of claim 25, wherein the analgesic agent is selected from the group consisting of lidocaine, pramoxine, benzocaine, hydrocortisone, and a combination thereof.

33. The method of claim 25, wherein the antipruritic agent is selected from the group consisting of mint oil, menthol, camphor, Hydroxyphenyl Propamidobenzoic Acid, Avena sativa (Oat) Kernel Extract, Beta Glucan, an Avenanthramide, and combinations thereof.

34. The method of claim 25, wherein the antihistamine is diphenylhydramine.

35. The method of claim 25, wherein the macrolide selected from the group consisting of rapamycin, pimecrolimus, temsirolimus, everolimus, and tacrolimus.

36. The method of claim 25, wherein the macrolide is pimecrolimus.

37. The method of claim 25, wherein the additional active agent consists of pimecrolimus.

38. The method of any one of claims 1-23, wherein the topical pharmaceutical lotion does not include any additional active agents.

39. The method of any one of claims 1-23, wherein the topical pharmaceutical lotion consists of the therapeutically active vehicle.

40. A method for treating moderate to severe atopic dermatitis (AD) in a subject in need thereof, comprising administering to the affected area a topical pharmaceutical lotion comprising 0.5-1.5 wt. % pimecrolimus.

41. The method of claim 40 wherein the concentration of pimecrolimus is about 1 wt. %.

42. The method of claim 40 or 41 wherein the topical pharmaceutical lotion is in the form of an emulsion comprising an oil phase, a water phase, one or more viscosity modifying agents, optionally one or more preservatives, and optionally one or more buffers.

43. The method of any one of claims 40-42, wherein the topical pharmaceutical lotion is an emulsion lotion comprising:
an oil phase comprising:
1.0 wt. % Pimecrolimus;
10 wt. % to 15 wt. % Fatty alcohols selected from Oleyl Alcohol, Cetyl Alcohol, Stearyl Alcohol, and combinations thereof;
10 wt. % to 20 wt. % Medium-chain triglycerides; and
1 wt. % to 3 wt. % Mono-glycerides and Di-glycerides;
a viscosity modifying agent comprising 0.05 wt. % to 1 wt. % Carbomer Homopolymer Type A;
a water phase comprising:
0.5 wt. % to 2 wt. % Sodium Cetostearyl Sulfate; and
2 wt. % to 8 wt. % propylene glycol; and
25 wt. % to 40 wt. % Water;
one or more preservatives comprising 0.5 wt. % to 2 wt. % benzyl alcohol;
one or more buffers comprising:
0.025 wt. % to 0.2 wt. % Citric Acid; and
Sodium Hydroxide, quantum satis (q.s.) to pH 5-7.5.

44. The method of claim 43, wherein the oil phase comprises 15 wt. % Medium-chain triglycerides.

45. The method of claim 43 or 44, wherein the oil phase comprises 2 wt. % Mono-glycerides and Di-glycerides.

46. The method of claim 43-45, wherein the viscosity modifying agent comprises 0.2 wt. % Carbomer Homopolymer Type A.

47. The method of claim 43-46, wherein the water phase comprises 1 wt. % Sodium Cetostearyl Sulfate.

48. The method of claim 43-47, wherein the one or more preservatives comprise 1 wt. % benzyl alcohol.

49. The method of claim 43-48, wherein the one or more buffers comprise 0.05 wt. % citric acid.

50. The method of claim 43-49, wherein the Sodium Hydroxide is q.s. to pH 5.5.

51. The method of any one of claims 40-50, wherein the topical pharmaceutical composition is administered to the affected area twice daily.

52. The method of any one of claims 40-51, wherein the duration of treatment is at least four weeks.

53. The method of any one of claims 40-52, wherein the duration of treatment is six weeks.

54. The method of any one of claims 40-53, wherein the atopic dermatitis is severe atopic dermatitis.

55. The method of any one of claims 40-54, wherein the subject at the start of treatment exhibits moderate to severe AD, characterized by (a) clearly perceptible erythema (dull red), clearly perceptible induration/papulation, and/or clearly perceptible lichenification, wherein oozing or crusting may be present; or (b) marked erythema (deep or bright red), marked induration/papulation, and/or marked lichenification, wherein the disease is wide-spread in extent, and wherein oozing or crusting may be present.

56. The method of any one of claims 40-55, wherein the subject at the start of treatment exhibits severe AD, characterized by marked erythema (deep or bright red), marked induration/papulation, and/or marked lichenification, wherein the disease is wide-spread in extent, and wherein oozing or crusting may be present.

57. The method of any one of claims 40-56, wherein the subject at the start of treatment has an Investigator Global Assessment (IGA) Scale score of greater than 3.

58. The method of any one of claims 40-57, wherein the subject at the start of treatment has an EASI score of greater than 21.

59. The method of any one of claims 40-58, wherein the subject exhibits at least a 2-grade reduction in the Investigator Global Assessment (IGA) Scale and exhibits an IGA that equates to “Clear” (0) or “Almost Clear” (1) after at least four weeks of treatment.

60. The method of claim 40-58, wherein the subject exhibits at least a 2-grade reduction in the Investigator Global Assessment (IGA) Scale and exhibits an IGA that equates to “Clear” (0) or “Almost Clear” (1) after six weeks of treatment.

61. The method of any one of claims 40-59, wherein the subject exhibits 75% reduction in the Eczema Area Severity Index (EASI) score after at least four weeks of treatment.

62. The method of any one of claims 40-59, wherein the subject exhibits 75% reduction in the Eczema Area Severity Index (EASI) score after six weeks of treatment.

63. The method of any one of claims 40-61, wherein the subject exhibits at least a 1 grade improvement from baseline in the severity of pruritus score and a pruritus score equating to “none” or “mild” after at least one week of treatment.

64. The method of any one of claims 40-61, wherein the subject exhibits at least a 1 grade improvement from baseline in the severity of pruritus score and a pruritus score equating to “none” or “mild” after two weeks of treatment.

65. A topical pharmaceutical lotion comprising a therapeutically active vehicle, wherein the therapeutically active vehicle comprises:
an oil phase comprising:
10 wt. % to 15 wt. % Fatty alcohols selected from Oleyl Alcohol, Cetyl Alcohol, Stearyl Alcohol, and combinations thereof;
10 wt. % to 20 wt. % Medium-chain triglycerides; and
1 wt. % to 3 wt. % Mono-glycerides and Di-glycerides;
a viscosity modifying agent comprising 0.05 wt. % to 1 wt. % Carbomer Homopolymer Type A;
a water phase comprising:
0.5 wt. % to 2 wt. % Sodium Cetostearyl Sulfate; and
25 wt. % to 75 wt. % Water;
one or more preservatives comprising 0.5 wt. % to 2 wt. % benzyl alcohol;
one or more buffers comprising:
0.025 wt. % to 0.2 wt. % Citric Acid; and
Sodium Hydroxide, quantum satis (q.s.) pH 5-7.5.

66. The topical pharmaceutical lotion of claim 65, wherein the water phase further comprises 2 wt. % to 8 wt. % propylene glycol.

67. The topical pharmaceutical lotion of claim 65 or 67, wherein the oil phase comprises 15 wt. % Medium-chain triglycerides.

68. The topical pharmaceutical lotion of claims 65-68, wherein the oil phase comprises 2 wt. % Mono-glycerides and Di-glycerides.

69. The topical pharmaceutical lotion of claim 65-68, wherein the viscosity modifying agent comprises 0.2 wt. % Carbomer Homopolymer Type A.

70. The topical pharmaceutical lotion of claim 65-69, wherein the water phase comprises 1 wt. % Sodium Cetostearyl Sulfate.

71. The topical pharmaceutical lotion of claim 65-70, wherein the one or more preservatives comprise 1 wt. % benzyl alcohol.

72. The topical pharmaceutical lotion of claim 65-71, wherein the one or more buffers comprise 0.05 wt. % citric acid.

73. The topical pharmaceutical lotion of claim 65-72, wherein the Sodium Hydroxide is q.s. to pH 5.5.

74. The topical pharmaceutical lotion of claims 65-73, wherein the topical pharmaceutical lotion is an emulsion.

75. The topical pharmaceutical lotion of claims 65-74, wherein the topical pharmaceutical lotion comprises an additional active agent.

76. The topical pharmaceutical lotion of any one of claims 65-75, wherein the topical pharmaceutical lotion comprises about 1-1.5 wt. % of the additional active agent.

77. The topical pharmaceutical lotion of claim 75 or 76, wherein the additional active agent is selected from the group consisting of an AD agent, a natural moisturizing factor (NMF), a peptide/collagen synthesis agent, a skin rejuvenation agent, an acne agent, a skin protectant, an analgesic agent, an antipruritic agent, an antihistamine, a macrolide, and combinations thereof.

78. The topical pharmaceutical lotion of claim 77, wherein the AD agent is Ophiopogon japonicus (Mondo grass).

79. The topical pharmaceutical lotion of claim 77, wherein the NMF may be selected from the group consisting of pyrrolidone carboxylic acid (PCA), hyaluronic acid, a ceramide, lactic acid, an amino acid, urea, squalene, and a combination thereof.

80. The topical pharmaceutical lotion of claim 77, wherein the peptide/collagen synthesis agent is selected from the group consisting of Palmitoyl Tripeptide-38, Palmitoyl Tripeptide-1, Palmitoyl Tetrapeptide-7, Acetyl hexapeptide 3, Acetyl hexapeptide 8, Acetyl hexapeptide 20, Palmitoyl oligopeptide, Lupine protein, a fractionated peptide extract (e.g., green peptides), and a combination thereof.

81. The topical pharmaceutical lotion of claim 77, wherein the skin rejuvenation agent is selected from the group consisting of a retinol, Retinyl Palmitate, Hydroxypinacolone Retinoate, Retinyl Lenoleate, bakuchiol extract (natural and green retinol alternative), Ceratonia siliqua (Locust bean gum), an Alpha Hydroxy Acid, a Beta Hydroxy Acid, and a combination thereof.

82. The topical pharmaceutical lotion of claim 77, wherein the acne agent is selected from the group consisting of Punica granatum Pericarp Extract (pomegranate peel extract), Tea tree oil, Salicylic acid, benzoyl peroxide (BPO), sulfur, and a combination thereof.

83. The topical pharmaceutical lotion of claim 77, wherein the skin protectant is selected from the group consisting of allantoin, calamine, and a combination thereof.

84. The topical pharmaceutical lotion of claim 77, wherein the analgesic agent is selected from the group consisting of lidocaine, pramoxine, benzocaine, hydrocortisone, and a combination thereof.

85. The topical pharmaceutical lotion of claim 77, wherein the antipruritic agent is selected from the group consisting of mint oil, menthol, camphor, Hydroxyphenyl Propamidobenzoic Acid, Avena sativa (Oat) Kernel Extract, Beta Glucan, an Avenanthramide, and combinations thereof.

86. The topical pharmaceutical lotion of claim 77, wherein the antihistamine is diphenylhydramine.

87. The topical pharmaceutical lotion of claim 77, wherein the macrolide selected from the group consisting of rapamycin, pimecrolimus, temsirolimus, everolimus, and tacrolimus.

88. The topical pharmaceutical lotion of claim 77, wherein the macrolide is pimecrolimus.

89. The topical pharmaceutical lotion of claim 77, wherein the additional active agent consists of pimecrolimus.

90. The topical pharmaceutical lotion of claim 65 or 66, wherein the topical pharmaceutical lotion does not include any additional active agents.

91. The topical pharmaceutical lotion of claim 65 or 66, wherein the topical pharmaceutical lotion consists of the therapeutically active vehicle.
